(19) United States
(12) Patent Application Publication
(10) Pub. No.: US 2024/0352086 A1
(43) Pub. Date: Oct. 24, 2024

(54) COMPOSITIONS AND METHODS COMPRISING LIPID ASSOCIATED TRANSMEMBRANE DOMAINS

(71) Applicant: The Regents of the University of California, Oakland, CA (US)

(72) Inventors: Kira Podolsky, San Diego, CA (US); Neal Devaraj, San Diego, CA (US)

(21) Appl. No.: 18/685,350

(22) PCT Filed: Aug. 23, 2022
(86) PCT No.: PCT/US2022/075366

§ 371 Date: Jul. 13, 2024

Related U.S. Application Data
(60) Provisional application No. 63/236,576, filed on Aug. 24, 2021.

(51) Int. Cl. C07K 14/705

(52) U.S. Cl. CPC C07K 14/705; C07K 2319/03; C07K 2319/92

(57) ABSTRACT

Provided herein, inter alia, are compositions and methods including transmembrane domains comprising a split intein and vesicles including transmembrane domains with a split intein. In embodiments, methods for generating vesicle embedded proteins in vitro without the use of denaturing agents, and their compositions, are provided.

### CROSS-REFERENCE TO RELATED APPLICATIONS

This application claims priority to U.S. Provisional Application No. 63/236,576, filed Aug. 24, 2021, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. 1935372 awarded by the National Science Foundation. The government has certain rights in the invention.

### SEQUENCE LISTING

The material in the accompanying Sequence Listing is hereby incorporated by reference in its entirety. The accompanying file, named “048537-649001WO_SL_ST26.xml” was created on Aug. 23, 2022 and is 11,050 bytes. The file can be accessed using Microsoft Word on a computer that uses Windows OS.

### BACKGROUND OF THE INVENTION

A transmembrane protein is a type of integral membrane protein that spans the entirety of the cell membrane. These transmembrane proteins contain one or more membrane-spanning domains as well as domains, from four to several hundred residues long, extending into the aqueous medium on each side of the bilayer. In all the transmembrane proteins examined to date, the membrane-spanning domains are a helices or multiple 3 strands. In contrast, some integral proteins are anchored to one of the membrane leaflets by covalently bound fatty acids. In these proteins, the bound fatty acid is embedded in the membrane, but the polypeptide chain does not enter the phospholipid bilayer.

Cellular lipid membranes are embedded with transmembrane proteins crucial to cell function. Elucidating membrane proteins' diverse structures and biophysical mechanisms is increasingly necessary due to their growing prevalence as a therapeutic target and sheer ubiquity in cells. Most biophysical characterization strategies of transmembrane proteins rely on the tedious overexpression and isolation of recombinant proteins and their reconstitution in model phospholipid bilayers. Unfortunately, membrane protein reconstitution depends on the use of denaturing and unnatural detergents that may interfere with protein structure and function.

Hence, there is an unmet need for efficient approaches for expression and reconstitution of functional membrane proteins.

### BRIEF SUMMARY OF THE INVENTION

In an aspect is provided a transmembrane domain covalently bound to a first intein of a split intein pair, wherein the transmembrane domain is embedded within a phospholipid layer.

In another aspect is provided a transmembrane domain provided herein including embodiments thereof, wherein the transmembrane domain is covalently bound to the first intein through a covalent linker.

In another aspect is provided a fusion protein including a transmembrane domain covalently bound to a biologically active protein domain through a first peptide linker, wherein the transmembrane domain is embedded within a phospholipid layer; and wherein the first peptide linker includes an intein scar amino acid sequence.

In another aspect is provided a method of synthesis of a fusion protein, the method including: (a) contacting a transmembrane domain with a biologically active protein domain, wherein the transmembrane domain is covalently bound to a first intein of a split intein pair and the transmembrane domain is embedded within a phospholipid layer, wherein the biologically active protein domain is covalently bound to a second intein of the split intein pair, and (b) allowing the first intein to react with the second intein thereby forming the fusion protein.

In another aspect is provided a kit composition including a transmembrane domain covalently bound to a first intein of a split intein pair, wherein the transmembrane domain is embedded within a phospholipid layer.

In an aspect, provided herein are methods of synthesis of a transmembrane polypeptide comprising contacting a first polypeptide comprising a transmembrane domain of the transmembrane polypeptide covalently bound to a C-intein with a second polypeptide covalently bound to an N-intein or contacting the first polypeptide comprising the transmembrane domain covalently bound to a N-intein with the second polypeptide covalently bound to an C-intein. In aspects, the method further includes reconstituting the first polypeptide in a vesicle.

### BRIEF DESCRIPTION OF THE DRAWINGS

FIGS. 1A-1B show semisynthetic split intein-mediated ligation. FIG. 1: Cartoon schematic of the steps of semisynthesis in giant unilamellar vesicles (GUVs) is shown from synthesis to reconstitution to ligation. The model soluble protein of interest, green fluorescent protein (GFP; green), fused to the Cfa^{N} split intein domain was expressed in E. coli, while the Transmembrane (TM) peptide, WALP), fused to the Cfa^{C} split intein domain was fabricated via solid-phase peptide synthesis (SPPS). Cfa^{C}-WALP was reconstituted into GUVs in randomly distributed orientations within the membrane. Upon addition of the soluble GFP-Cfa^{N} construct to peptide-loaded GUVs, the split intein-mediated ligation occurs to produce TM semisynthetic GFP-WALP embedded GUVs. See FIG. 2 FIG. 1B. Single amino acid sequences of both the Cfa^{N} (SEQ ID NO:11) and Cfa^{C} (SEQ ID NO:12) inteins and their respective protein and peptide fusions. The dashed lines represent glycine linkers. The asterisks denote the position of the cyanine-based fluorescent (CF) fluorophore that is conjugated to a lysine side chain of the peptide.

FIGS. 2A-2B show transmembrane peptide reconstitution into phospholipid membranes. FIG. 2A: Brightfield and fluorescence (488 nm) images of a hydrated 1,2-dioleoyl-sn-glycero-3-phosphatidylcholine (DOPC) vesicle containing Cfa^{C}-WALP-CF. Scale bar 10 m. FIG. 2B: CD spectra of 20 μM Cfa^{C}-WALP in water and reconstituted into SUVs in water.

FIGS. 3A-3D show semisynthetic split intein-mediated ligation occurs in vesicle and GUV membranes. FIG. 3A: Chromatogram of a liquid chromatography-electrospray ionization-time-of-flight mass spectrometry (LC-ESI-TOFMS) run of the reaction between GFP-Cfa^{N}-His₆, E, and Cfa^{C}-WALP, G, in vesicles. Each peak corresponds to a reactant, intermediate, or product that is listed in FIG. 3B with their corresponding calculated molecular weight (MW) and experimental ESI MW. FIG. 3C: SDS-PAGE gel of the reaction in FIG. 3A. Lanes 2-4 are the reaction between E and G, lanes 5-7 is E only, and lanes 8-10 are G only. The GFP-WALP product, F, is highlighted in boxes throughout the figure. FIG. 3D: Confocal micrographs show that GFP does not bind to DOPC GUVs alone (upper row) but does bind to Cfa^{C}-WALP incorporated DOPC GUVs after 24 h (lower row). GFP channel=488 nm. Scale bar, 5 m.

FIGS. 4A-4D show building a functional semisynthetic transmembrane protein in GUVs. FIG. 4A: A cartoon representation depicts the fluorescent (asterisks) synthetic transmembrane peptide fused to the extracellular domain of fluorescently labeled Programmed cell death protein 1 (PD-1). FIG. 4B: Brightfield and fluorescent micrographs of the semisynthetic JF-PD-1-WALP-CF transmembrane product in a GUV. The CF channel=488 nm. JF 646=638 nm. FIG. 4C: Cartoon schematic of the microcluster experiment where large surface of a GUV contacts a SLB due to the enrichment of PD-1 at the GUV/supported lipid bilayer (SLB) interface due to PD-1/PD-L1 binding. FIG. 4D: Total Internal Reflection Fluorescence (TIRF) brightfield and fluorescence micrographs of the SLB/GUV interface showing enrichment of fluorescent peptide and PD-1 signals at the interface. In the presence of PD-1 blockade (bottom row), there is no enrichment of either signal although a GUV remains present at the SLB surface.

FIG. 5 shows a reaction scheme of the general mechanism of the split intein-mediated protein ligation, or protein trans-splicing events. The Cfa domains (blue and yellow) of GFP-Cfa^{N}-His₆ and Cfa^{C}-WALP associate noncovalently. An N to S acyl shift and subsequent transthioesterification results in a branched intermediate formation where GFP, WALP, and the Cfa^{C} are covalently linked while the Cfa^{N} is noncovalently associated. Succinimide formation results in the loss of both split inteins, and an S to N acyl shift between the proteins of interest results in a native peptide bond between GFP and WALP.

### DETAILED DESCRIPTION OF THE INVENTION

Provided herein are methods and compositions for generating phospholipid layer-associated polypeptides that include a transmembrane domain without the use of denaturing conditions. In aspects, provided herein are methods and compositions including transmembrane domains comprising a first split intein of a split intein pair and vesicles including transmembrane domains with a first split intein of a split intein pair.

### I. Definitions

Before the present invention is further described, it is to be understood that this invention is not strictly limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N—hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is

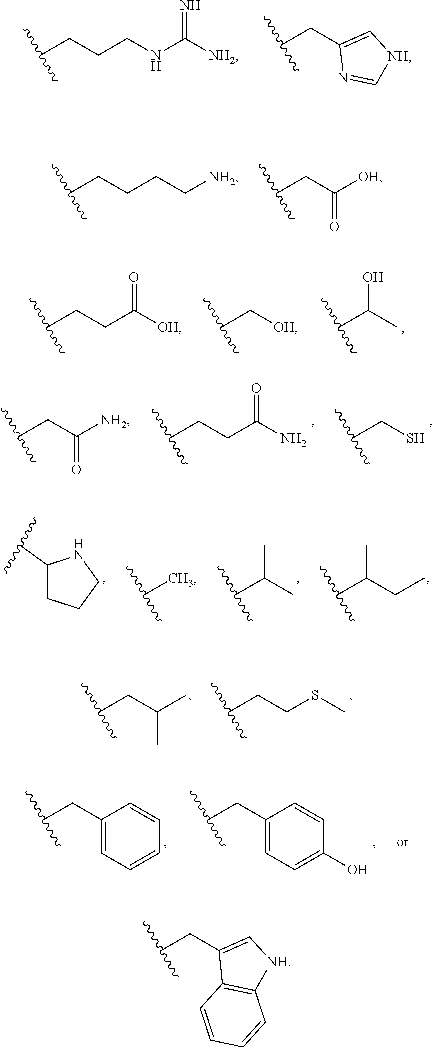

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride,cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride,2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)-OH, Boc-Phe(4-Br)-OH, Boc-D-Phe(4-Br)-OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)-OH, Fmoc-Phe(4-Br)-OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As used herein, the term “linker” or “peptide linker” is used in accordance with its plain ordinary meaning and refers to peptide used to bind or link two molecules of interest together. The linker may usually be rich in glycine for flexibility, as well as serine or threonine for solubility. The linker can either connect the N-terminus of a first molecule with the C-terminus of a second molecule, or vice versa.

As used herein, the term “transmembrane protein” is used in accordance with its plain ordinary meaning and refers to a type of integral membrane protein that spans the entirety of the cell membrane. Many transmembrane proteins function as gateways to permit the transport of specific substances across the membrane. They frequently undergo significant conformational changes to move a substance through the membrane. They are usually highly hydrophobic and aggregate and precipitate in water. They require detergents or nonpolar solvents for extraction, although some of them (beta-barrels) may be also extracted using denaturing agents.

As used herein, the term “transmembrane domain” is used in accordance with its plain ordinary meaning and refers to a region of a protein that spans or resides in a phospholipid bilayer. A transmembrane domain is largely comprised of hydrophobic amino acids and facilitates the anchorage of a membrane protein to cellular lipid membranes. In embodiments, the topological conformation of a transmembrane domain is an alpha helix. In embodiments, the topological conformation of a transmembrane domain is a beta barrel.

As used herein, the term “WALP peptide” is used in accordance with its plain and ordinary meaning and refers to a polypeptide comprising tryptophan (W), alanine (A), and leucine (L) amino acids that typically form an alpha helix. WALP peptides are useful for studying the properties of proteins in lipid membranes such as orientation, extent of insertion and hydrophobic mismatch.

As used herein, the term “semisynthesis” is used in accordance with its plain ordinary meaning and refers to a type of chemical synthesis that uses chemical compounds isolated from natural sources (such as microbial cell cultures or plant material) as the starting materials to produce other novel compounds with distinct chemical and medicinal properties. The novel compounds generally have a high molecular weight or a complex molecular structure, more so than those produced by total synthesis from simple starting materials. Semisynthesis is a means of preparing many medicines more cheaply than by total synthesis since fewer chemical steps are necessary. Here, semisynthesis includes transmembrane proteins.

As used herein, the term “lipid” is used in accordance with its plain ordinary meaning and refers to a micro biomolecule that is soluble in non-polar solvents. Non-polar solvents are typically hydrocarbons used to dissolve other naturally occurring hydrocarbon lipid molecules that do not (or do not easily) dissolve in water, including fatty acids, waxes, sterols, fat-soluble vitamins (such as vitamins A, D, E, and K), monoglycerides, diglycerides, triglycerides, and phospholipids. The functions of lipids include storing energy, signaling, and acting as structural components of cell membranes. Lipids have applications in the cosmetic and food industries as well as in nanotechnology.

As used herein, the term “lipid bilayer” or “phospholipid bilayer” is used in accordance with its plain ordinary meaning and refers to a polar membrane made of two layers of lipid molecules. These lipid bilayers are flat sheets that can form a continuous barrier around cells. Phospholipid bilayers are composed of amphiphilic phospholipids that have a hydrophilic phosphate head group and a hydrophobic tail consisting of two fatty acid chains. The phosphate head group of a phospholipid can alter the surface chemistry of the bilayer. In addition, the fatty acid tails can affect membrane properties (e.g. phase of the bilayer).

As used herein, the term “liposome” is used in accordance with its plain ordinary meaning and refers to a spherical vesicle having at least one lipid bilayer. The liposome can be used as a drug delivery vehicle for administration of nutrients and pharmaceutical drugs, such as lipid nanoparticles in mRNA vaccines, and DNA vaccines. Liposomes can be prepared by disrupting biological membranes (such as by sonication). Liposomes are most often composed of phospholipids, especially phosphatidylcholine, but may also include other lipids, such as egg phosphatidylethanolamine, so long as they are compatible with lipid bilayer structure. A liposome design may employ surface ligands for attaching to unhealthy tissue. The major types of liposomes are the multilamellar vesicle (MLV, with several lamellar phase lipid bilayers), the small unilamellar liposome vesicle (SUV, with one lipid bilayer), the large unilamellar vesicle (LUV), and the cochleate vesicle. A multivesicular liposome is a vesicle that contains one or more smaller vesicles. Liposomes should not be confused with lysosomes, or with micelles and reverse micelles composed of monolayers.

As used herein, the term “vesicles” or “lipid vesicles” is used in accordance with its plain ordinary meaning and refers to a structure within or outside a cell, consisting of liquid or cytoplasm enclosed by a lipid bilayer. The vesicles form naturally during the processes of secretion (exocytosis), uptake (endocytosis) and transport of materials within the plasma membrane. Alternatively, they may be prepared artificially, in which case they are called liposomes (not to be confused with lysosomes). If there is only one phospholipid bilayer, they are called unilamellar liposome vesicles; otherwise they are called multilamellar. The membrane enclosing the vesicle is also a lamellar phase, similar to that of the plasma membrane, and intracellular vesicles may fuse with the plasma membrane to release their contents outside the cell. The vesicles may also fuse with other organelles within the cell. A vesicle released from the cell is known as an extracellular vesicle. The vesicles perform a variety of functions. Because it is separated from the cytosol, the inside of the vesicle may be made to be different from the cytosolic environment. For this reason, the vesicles are a basic tool used by the cell for organizing cellular substances. The vesicles are involved in metabolism, transport, buoyancy control, and temporary storage of food and enzymes. The vesicles may also act as chemical reaction chambers.

As used herein, the term “giant unilamellar vesicles” is used in accordance with its plain ordinary meaning and refers to a simple model membrane system of cell-size, which are instrumental in studying the function of more complex biological membranes involving heterogeneities in lipid composition, shape, mechanical properties, and chemical properties.

As used herein, the term “nanodisc” is used in accordance with its plain ordinary meaning and refers to a discoidal protein in which the hydrophobic edge of a phospholipid bilayer is surrounded by amphipathic molecules (e.g. proteins, peptides and synthetic polymers). Nanodiscs are useful for studying membrane proteins because they can solubilize and stabilize membrane proteins and represent a more native environment than liposomes and micelles.

As used herein, the term “biorthogonality” is used in accordance with its plain ordinary meaning and refers to a any chemical reaction that may occur inside of living systems without interfering with native biochemical processes. The use of bioorthogonal chemistry typically proceeds in two steps. First, a cellular substrate is modified with a bioorthogonal functional group (chemical reporter) and introduced to the cell; substrates include metabolites, enzyme inhibitors, etc. The chemical reporter must not alter the structure of the substrate dramatically to avoid affecting its bioactivity. Secondly, a probe containing a complementary functional group is introduced to react and label the substrate.

As used herein, the term “chemoselectivity” is used in accordance with its plain ordinary meaning and refers to a term that describes the ability of a reagent or inter-mediate to react with one group or atom in a mole-cule in preference to another group or atom present in the same molecule. For example, chemoselective reaction also may occur when a carbohydrate radical reacts with another mole-cule present in the reaction mixture.

As used herein, the term “phospholipid” is used in accordance with its plain ordinary meaning and refers to a class of lipids whose molecule has a hydrophilic “head” containing a phosphate group, and two hydrophobic “tails” derived from fatty acids, joined by a glycerol molecule. Marine phospholipids typically have omega-3 fatty acids EPA and DHA integrated as part of the phospholipid molecule. The phosphate group may be modified with simple organic molecules such as choline, ethanolamine or serine. Phospholipids are a key component of all cell membranes. They may form lipid bilayers because of their amphiphilic characteristic. In eukaryotes, cell membranes also contain another class of lipid, sterol, interspersed among the phospholipids. The combination provides fluidity in two dimensions combined with mechanical strength against rupture. Purified phospholipids are produced commercially and have found applications in nanotechnology and materials science.

As used herein, the term “expression” is used in accordance with its plain ordinary meaning and refers to a step involved in the production of the polypeptide including, but not limited to, transcription, post-transcriptional modification, translation, post-translational modification, and secretion. Expression may be detected using conventional techniques for detecting protein (e.g., ELISA, Western blotting, flow cytometry, immunofluorescence, immunohistochemistry, etc.).

As used herein, the term “PD-1” or “PD-1 protein” is used in accordance with its plain ordinary meaning and refers to a recombinant or naturally-occurring forms of the Programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In some aspects, the variants or homologs have at least 90%, 95%, 96%, 97%, 98%, 99% or 100% amino acid sequence identity across the whole sequence or a portion of the sequence (e.g. a 50,100,150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1” or “PD-L1 protein” as referred to herein includes any of the recombinant or naturally-occurring forms of programmed death ligand 1 (PD-L1) also known as cluster of differentiation 274 (CD 274) or variants or homologs thereof that maintain PD-L1 activity (e.g. within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto.

As used herein, the term “EGFR” or “EGFR protein” is used in accordance with its plain ordinary meaning and includes any of the recombinant or naturally-occurring forms of epidermal growth factor receptor, also known as Proto-oncogene c-ErbB-1, Receptor tyrosine-protein kinase erbB-1, ERBB, ERBB1, HER1, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

As used herein, the term “proteorhodopsin” or “proteorhodopsin protein” is used in accordance with its plain ordinary meaning and refers to a member of the proteorhodopsin family of transmembrane proteins that use retinal as a chromophore for light-mediated functionality. Proteorhodopsin includes any of the recombinant or naturally-occurring forms of proteorhodopsin proteins, also known as pRhodopsins, or variants or homologs thereof that maintain proteorhodopsin activity (e.g. within at least 50%, 80%, 90%, 95%, 96%, 97%, 98%, 99% or 100% activity compared to a proteorhodopsin protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring proteorhodopsin protein.

As used herein, the term “receptor tyrosine kinase” or “receptor tyrosine kinase protein” is used in accordance with its plain and ordinary meaning and refers to a member of the class of high-affinity cell surface receptors known as receptor tyrosine kinases. Receptor tyrosine kinases comprise an extracellular domain, a transmembrane domain, and an intracellular domain. The extracellular domain binds target ligands of interest to initiate intracellular signaling, whereas the intracellular domain is the catalytic domain, which has kinase activity. Receptor tyrosine kinase includes any of the recombinant or naturally-occurring forms of receptor tyrosine kinase proteins, also known as RTKs, or variants or homologs thereof that maintain receptor tyrosine kinase activity (e.g. within at least 50%, 80%, 90%, 95%, 96%, 97%, 98%, 99% or 100% activity compared to a receptor tyrosine kinase protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring receptor tyrosine kinase protein. In embodiments, the receptor tyrosine kinase is an EGFR protein.

As used herein, the term “notch receptors” or “notch receptor proteins” is used in accordance with its plain ordinary meaning and refers to members of the family of single-pass transmembrane domain receptor proteins that bind the ligand notch. Notch receptors includes any of the recombinant or naturally-occurring forms of notch receptor proteins or variants or homologs thereof that maintain notch receptor activity (e.g. within at least 50%, 80%, 90%, 95%, 96%, 97%, 98%, 99% or 100% activity compared to a notch receptor protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring notch receptor protein. In embodiments, the notch receptor protein is NOTCH1, NOTCH2, NOTCH3, or NOTCH4. In embodiments, the notch receptor protein is NOTCH1 and is substantially identical to the protein identified by the UniProt reference number P46531 or a variant or homolog having substantial identity thereto. In embodiments, the notch receptor protein is NOTCH2 and is substantially identical to the protein identified by the UniProt reference number Q04721 or a variant or homolog having substantial identity thereto. In embodiments, the notch receptor protein is NOTCH3 and is substantially identical to the protein identified by the UniProt reference number Q9UM47 or a variant or homolog having substantial identity thereto. In embodiments, the notch receptor protein is NOTCH4 and is substantially identical to the protein identified by the UniProt reference number Q99466 or a variant or homolog having substantial identity thereto.

As used herein, the term “hemagglutinin” or “hemagglutinin protein” is used in accordance with its plain ordinary meaning and refers to members of the family of receptor-binding membrane fusion glycoproteins produced by Paramyxoviridae viruses. Hemagglutinins recognize cell-surface glycoproteins containing sialic acid on the surface of host red blood cells and use them to enter the endosome of host cells. Hemagglutinin includes any of the recombinant or naturally-occurring forms of hemagglutinin proteins or variants or homologs thereof that maintain hemagglutinin activity (e.g. within at least 50%, 80%, 90%, 95%, 96%, 97%, 98%, 99% or 100% activity compared to a hemagglutinin protein). In some aspects, the variants or homologs have at least 90%, 95%, 96%, 97%, 98%, 99% or 100% amino acid sequence identity across the whole sequence or a portion of the sequence (e.g. a 50,100,150 or 200 continuous amino acid portion) compared to a naturally occurring hemagglutinin protein. In embodiments, the hemagglutinin is an influenza hemagglutinin, a measles hemagglutinin, a parainfluenza hemagglutinin, a mumps hemagglutinin, or a phytohaemagglutinin.

As used herein, the term “neuraminidase” or “neuraminidase protein” is used in accordance with its plain ordinary meaning and refers to a member of the family of glycoside hydrolase enzymes that cleave the glycosidic linkages of neuraminic acids. Neuraminidase includes any of the recombinant or naturally-occurring forms of neuraminidase proteins or variants or homologs thereof that maintain neuraminidase activity (e.g. within at least 50%, 80%, 90%, 95%, 96%, 97%, 98%, 99% or 100% activity compared to a neuraminidase protein). In some aspects, the variants or homologs have at least 90%, 95%, 96%, 97%, 98%, 99% or 100% amino acid sequence identity across the whole sequence or a portion of the sequence (e.g. a 50,100,150 or 200 continuous amino acid portion) compared to a naturally occurring neuraminidase protein. In embodiments, the neuraminidase is an exo-α-sialidase or an endo-α-sialidase.

As used herein, the term “ACE-2”, “ACE-2 protein” or “angiotensin converting enzyme 2” is used in accordance with its plain ordinary meaning and refer to any of the recombinant or naturally-occurring forms of the ACE2 enzyme, or variants or homologs thereof that maintain ACE-2 enzyme activity (e.g. within at least 50%, 80%, 90%, 95%, 96%, 97%, 98%, 99% or 100% activity compared to ACE-2). In some aspects, the variants or homologs have at least 90%, 95%, 96%, 97%, 98%, 99% or 100% amino acid sequence identity across the whole sequence or a portion of the sequence (e.g. a 50,100,150 or 200 continuous amino acid portion) compared to a naturally occurring ACE-2 protein. In embodiments, the ACE-2 protein is substantially identical to the protein identified by the UniProt reference number Q9BYF1 or a variant or homolog having substantial identity thereto.

As used herein, the term “rhomboid protease” or “rhomboid protease enzyme” is used in accordance with its plain ordinary meaning and refers to a member of the family of intramembrane protease enzymes which have active sites located within the phospholipid bilayer of cell membranes. Rhomboid protease includes any of the recombinant or naturally-occurring forms of neuraminidase proteins or variants or homologs thereof that maintain rhomboid protease activity (e.g. within at least 50%, 80%, 90%, 95%, 96%, 97%, 98%, 99% or 100% activity compared to a rhomboid protease enzyme). In some aspects, the variants or homologs have at least 90%, 95%, 96%, 97%, 98%, 99% or 100% amino acid sequence identity across the whole sequence or a portion of the sequence (e.g. a 50,100,150 or 200 continuous amino acid portion) compared to a naturally occurring rhomboid protease protein.

As used herein, the term “intein” is used in accordance with its plain ordinary meaning and refers to an amino acid sequence of a precursor protein that is removed in a protein splicing reaction. For example, in protein splicing inteins are removed from the precursor polypeptide with a ligation of the C-terminal and N-terminal ends of the excision site thereby forming a peptide bond. The two amino acid sequences joined together through the C-terminal and N-terminal ends are referred to as the C-extein and N-extein. Thus, the precursor protein may include an N-extein amino acid sequence attached to the intein amino acid sequence, which is in turn attached to the C-extein amino acid sequence.

Exteins can be either an N-extein or a C-extein depending on whether it is N-terminal or C-terminal to the intein. The extein can be any polypeptide. In embodiments the polypeptide includes a transmembrane domain, an extracellular domain, or an intracellular domain.

As used herein, the term “split inteins” or “split intein pair” is used in accordance with its plain ordinary meaning and refers to two separate polypeptides that can function as an intein in trans. The split intein pair includes one member of the split intein pair that includes the N-intein amino acid sequence (referred to herein a the “N-intein split pair member”) and the other member of the split intein pair that includes the C-intein amino acid sequence (referred to herein as the “C-intein split pair member”). In embodiments, both the N-intein split pair member and the C-intein split pair member include a portion of the intein amino acid sequence such that the aggregate of the split intein pair includes the full intein sequence. In embodiments, the N-intein and C-intein spontaneously assemble non-covalently and ligate the two exteins in trans. As used herein, a first intein of a split intein refers to either the N-intein and a C-intein of a split intein pair and a second interin of a split intein refers to either the corresponding C-intein and a N-intein of the split intein.

As used herein, the term “N-intein” when used in the context of the invention disclosed herein may be used synonymously with a N-intein split pair member. In embodiments, this N-intein split pair member is covalently linked to an N-extein and upon contacting its corresponding C-intein it facilitates the ligation of an N-extein and a C-extein.

As used herein, the term “C-intein” when used in the context of the invention disclosed herein may be used synonymously with a C-intein split pair member. In embodiments, the C-intein split pair member is covalently linked to a C-extein and upon assembling with an N-intein it facilitates the ligation of an N-extein to a C-extein.

As used herein, the term “intein scar” refers to one or more amino acids derived from an intein amino acid sequence that remains in the product peptide (i.e. the product peptide resulting from protein splicing of the precursor peptide). In embodiments, these intein scar amino acids result from the biochemical product of split intein ligation and/or from incorporation of unnatural linker amino acids to facilitate split intein ligation.

As used herein, the term “nanoparticle” is used in accordance with its plain ordinary meaning and refers to a particle wherein the longest diameter is less than or equal to 1000 nanometers. Nanoparticles may be composed of any appropriate material (e.g. lipids).

As used herein, the term “thioesterification reaction” is used in accordance with its plain ordinary meaning and refers to an intermediate reaction step in which a split intein pair ligates two exteins together to form a thioester.

As used herein, the term “biologically active protein domain” is used in accordance with its plain ordinary meaning and refers to a region of a protein that affects genes, proteins, or biological processes (e.g. catalytic domain).

As used herein, the term “polymersome” is used in accordance with its plain and ordinary meaning and refers to a class of artificial vesicles that can include amphiphilic synthetic block copolymers to form the vesicle membrane, and have radii ranging from 50 nm to 5 μm or more.

### II. Compositions

In an aspect is provided a transmembrane domain covalently bound to a first intein of a split intein pair, wherein the transmembrane domain is embedded within a phospholipid layer.

In embodiments, the transmembrane domain has a length of about 15 to about 200 amino acid residues. In embodiments, the transmembrane domain has a length of about 20 to about 200 amino acid residues. In embodiments, the transmembrane domain has a length of about 30 to about 200 amino acid residues. In embodiments, the transmembrane domain has a length of about 40 to about 200 amino acid residues. In embodiments, the transmembrane domain has a length of about 50 to about 200 amino acid residues. In embodiments, the transmembrane domain has a length of about 60 to about 200 amino acid residues. In embodiments, the transmembrane domain has a length of about 70 to about 200 amino acid residues. In embodiments, the transmembrane domain has a length of about 80 to about 200 amino acid residues. In embodiments, the transmembrane domain has a length of about 90 to about 200 amino acid residues. In embodiments, the transmembrane domain has a length of about 100 to about 200 amino acid residues. In embodiments, the transmembrane domain has a length of about 110 to about 200 amino acid residues. In embodiments, the transmembrane domain has a length of about 120 to about 200 amino acid residues. In embodiments, the transmembrane domain has a length of about 130 to about 200 amino acid residues. In embodiments, the transmembrane domain has a length of about 140 to about 200 amino acid residues. In embodiments, the transmembrane domain has a length of about 150 to about 200 amino acid residues. In embodiments, the transmembrane domain has a length of about 160 to about 200 amino acid residues. In embodiments, the transmembrane domain has a length of about 170 to about 200 amino acid residues. In embodiments, the transmembrane domain has a length of about 180 to about 200 amino acid residues. In embodiments, the transmembrane domain has a length of about 190 to about 200 amino acid residues.

In embodiments, the transmembrane domain has a length of about 15 to about 190 amino acid residues. In embodiments, the transmembrane domain has a length of about 15 to about 180 amino acid residues. In embodiments, the transmembrane domain has a length of about 15 to about 170 amino acid residues. In embodiments, the transmembrane domain has a length of about 15 to about 160 amino acid residues. In embodiments, the transmembrane domain has a length of about 15 to about 150 amino acid residues. In embodiments, the transmembrane domain has a length of about 15 to about 140 amino acid residues. In embodiments, the transmembrane domain has a length of about 15 to about 130 amino acid residues. In embodiments, the transmembrane domain has a length of about 15 to about 120 amino acid residues. In embodiments, the transmembrane domain has a length of about 15 to about 110 amino acid residues. In embodiments, the transmembrane domain has a length of about 15 to about 100 amino acid residues. In embodiments, the transmembrane domain has a length of about 15 to about 90 amino acid residues. In embodiments, the transmembrane domain has a length of about 15 to about 80 amino acid residues. In embodiments, the transmembrane domain has a length of about 15 to about 70 amino acid residues. In embodiments, the transmembrane domain has a length of about 15 to about 60 amino acid residues. In embodiments, the transmembrane domain has a length of about 15 to about 50 amino acid residues. In embodiments, the transmembrane domain has a length of about 15 to about 40 amino acid residues. In embodiments, the transmembrane domain has a length of about 15 to about 30 amino acid residues. In embodiments, the transmembrane domain has a length of about 15 to about 20 amino acid residues.

In embodiments, the transmembrane domain has a length of 15 to 200 amino acid residues. In embodiments, the transmembrane domain has a length of 20 to 200 amino acid residues. In embodiments, the transmembrane domain has a length of 30 to 200 amino acid residues. In embodiments, the transmembrane domain has a length of 40 to 200 amino acid residues. In embodiments, the transmembrane domain has a length of 50 to 200 amino acid residues. In embodiments, the transmembrane domain has a length of 60 to 200 amino acid residues. In embodiments, the transmembrane domain has a length of 70 to 200 amino acid residues. In embodiments, the transmembrane domain has a length of 80 to 200 amino acid residues. In embodiments, the transmembrane domain has a length of 90 to 200 amino acid residues. In embodiments, the transmembrane domain has a length of 100 to 200 amino acid residues. In embodiments, the transmembrane domain has a length of 110 to 200 amino acid residues. In embodiments, the transmembrane domain has a length of 120 to 200 amino acid residues. In embodiments, the transmembrane domain has a length of 130 to 200 amino acid residues. In embodiments, the transmembrane domain has a length of 140 to 200 amino acid residues. In embodiments, the transmembrane domain has a length of 150 to 200 amino acid residues. In embodiments, the transmembrane domain has a length of 160 to 200 amino acid residues. In embodiments, the transmembrane domain has a length of 170 to 200 amino acid residues. In embodiments, the transmembrane domain has a length of 180 to 200 amino acid residues. In embodiments, the transmembrane domain has a length of 190 to 200 amino acid residues.

In embodiments, the transmembrane domain has a length of 15 to 190 amino acid residues. In embodiments, the transmembrane domain has a length of 15 to 180 amino acid residues. In embodiments, the transmembrane domain has a length of 15 to 170 amino acid residues. In embodiments, the transmembrane domain has a length of 15 to 160 amino acid residues. In embodiments, the transmembrane domain has a length of 15 to 150 amino acid residues. In embodiments, the transmembrane domain has a length of 15 to 140 amino acid residues. In embodiments, the transmembrane domain has a length of 15 to 130 amino acid residues. In embodiments, the transmembrane domain has a length of 15 to 120 amino acid residues. In embodiments, the transmembrane domain has a length of 15 to 110 amino acid residues. In embodiments, the transmembrane domain has a length of 15 to 100 amino acid residues. In embodiments, the transmembrane domain has a length of 15 to 90 amino acid residues. In embodiments, the transmembrane domain has a length of 15 to 80 amino acid residues. In embodiments, the transmembrane domain has a length of 15 to 70 amino acid residues. In embodiments, the transmembrane domain has a length of 15 to 60 amino acid residues. In embodiments, the transmembrane domain has a length of 15 to 50 amino acid residues. In embodiments, the transmembrane domain has a length of 15 to 40 amino acid residues. In embodiments, the transmembrane domain has a length of 15 to 30 amino acid residues. In embodiments, the transmembrane domain has a length of 15 to 20 amino acid residues.

In embodiments, the phospholipid layer is a lipid vesicle, a nanodisc, a lipid nanoparticle, or a polymersome. In embodiments, the phospholipid layer is a lipid vesicle. In embodiments, the phospholipid layer is a nanodisc. In embodiments, the phospholipid layer is a lipid nanoparticle. In embodiments, the phospholipid layer is a polymersome. In embodiments, the phospholipid layer forms part of a lipid vesicle, a nanodisc, a lipid nanoparticle, or a polymersome. In embodiments, the phospholipid layer forms part of a lipid vesicle. In embodiments, the phospholipid layer forms part of a nanodisc. In embodiments, the phospholipid layer forms part of a lipid nanoparticle. In embodiments, the phospholipid layer forms part of a polymersome.

In embodiments, the first intein is a C-intein or an N-intein. In embodiments, the first intein is a C-intein. In embodiments, the first intein is an N-intein. In embodiments, the split intein is a C-intein or an N-intein from one of the following inteins: Cfa, PhoRadA, RmaDnaB^{Δ286}, SspDnaB^{Δ275}, SspDnaX, TvoVMA, NpuDnaE, NpuDnaB^{Δ283}, SspGyrB, TerThyX, AceL-TerL, PchPRP8, PfuRIR1-1, Psp-GDBPol-1, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, CwaDnaE, Maer(NIES843)DnaE, Mcht(PCC7420)DnaE, MtuRecA^{Δ300}, NspDnaE, OliDnaE, Sel(PC7942)DnaE, Ssp(PCC7002)DnaE, TerDnaE-3, TelDnaE, TvuDnaE, NeqPol, or TerThyX^{Δ132}. Abbreviations of inteins: AceL-TerL, Ace lake terminase large subunit intein from unknown host; AovDnaE, DnaE intein from Aphanizomenon ovalisporum; AspDnaE, DnaE intein from Anabaena species; AvaDnaE, DnaE intein from Anabaena variabilis; Cfa, consensus fast DnaE intein sequence; Cra(CS505)DnaE, DnaE intein from Cylindrospermopsis raciborskii CS505; Csp(CCY00110)DnaE, DnaE intein from Cyanothece sp CCY00110; Csp(PCC8801)DnaE, DnaE intein from Cyanothece sp PCC8801; CwaDnaE, DnaE intein from Crocosphaera watsonii; gp41-1 and gp41-8, gp41 DNA helicase inteins from unknown host; IMPDH-1, IMPDH intein from unknown host; Maer(NIES843)DnaE, DnaE intein from Microcystis aeruginosa NIES843; Mcht(PCC7420)-2DnaE, DnaE intein from Microcoleus chthonoplastes sp PCC7420; MtuRecAD228/285/300, minimized RecA inteins from Mycobacterium tuberculosis; NeqPol, DNA polymerase intein from Nanoarchaeum equitans; NpuDnaBD283, minimized DnaB intein from Nostoc punctiforme; NpuDnaE, DnaE intein from Nostoc punctiforme; NrdJ, NrdJ intein from unknown host; NspDnaE, DnaE intein from Nostoc sp PCC7120; OliDnaE, DnaE intein from Oscillatoria limnetica; PchPRP8, PRP8 intein from Penicillium chrysogenum; PfuRIR1-1, RIR1 intein from Pyrococcus furiosus; PfuRIR1-2, RIR1 intein from Pyrococcus furiosus; PhoRadA, RadA intein from Pyrococcus horikoshii; Psp-GDBPol-1 DNA polymerase intein from Pyrococcus sp GB-D; RmaDnaBD271/D286, minimized DnaB inteins from Rhodothermus marinus; SceVMAD206/227, minimized VMA inteins from Saccharomyces cerevisiae; Sel(PC7942)DnaE, DnaE intein from Synechococcus elongatus PC7942; SspDnaBD274/275, minimized DnaB inteins from Synechocystis sp PCC6008; SspDnaBM86D275, M86 mutant of minimized DnaB intein from Synechocystis sp PCC6008; SspDnaE, DnaE intein from Synechocystis sp PCC6008; Ssp(PCC7002)DnaE, DnaE intein from Synechococcus sp PCC7002; SspDnaX, DnaX intein from Synechocystis sp PCC6008; SspGyrB, GyrB intein from Synechocystis sp PCC6008; TelDnaE, DnaE intein from Thermosynechococcus elongatus; TerDnaE-3, DnaE intein from Trichodesmium erythraeum; TerThyXD132, ThyX intein from Trichodesmium erythraeum; TvoVMA, VMA intein from Thermoplasma volcanium; TvuDnaE, DnaE intein from Thermosynechococcus vulcanus.

In embodiments, the split intein is a C-intein or an N-intein from PhoRadA. In embodiments, the split intein is a C-intein or an N-intein from RmaDnaB^{Δ286}. In embodiments, the split intein is a C-intein or an N-intein from SspDnaB^{Δ275}. In embodiments, the split intein is a C-intein or an N-intein from SspDnaX. In embodiments, the split intein is a C-intein or an N-intein from TvoVMA. In embodiments, the split intein is a C-intein or an N-intein from NpuDnaE. In embodiments, the split intein is a C-intein or an N-intein from NpuDnaB^{Δ283}. In embodiments, the split intein is a C-intein or an N-intein from SspGyrB. In embodiments, the split intein is a C-intein or an N-intein from TerThyX. In embodiments, the split intein is a C-intein or an N-intein from AceL-TerL. In embodiments, the split intein is a C-intein or an N-intein from PchPRP8. In embodiments, the split intein is a C-intein or an N-intein from PfuRIR1-1. In embodiments, the split intein is a C-intein or an N-intein from Psp-GDBPol-1. In embodiments, the split intein is a C-intein or an N-intein from PfuRIR1-2, SceVMA^{Δ206}. In embodiments, the split intein is a C-intein or an N-intein from RmaDnaB^{Δ271}. In embodiments, the split intein is a C-intein or an N-intein from MtuRec^{Δ285}. In embodiments, the split intein is a C-intein or an N-intein from SspDnaB^{Δ274}. In embodiments, the split intein is a C-intein or an N-intein from gp41-8. In embodiments, the split intein is from SceVMAA²²⁷. In embodiments, the split intein is a C-intein or an N-intein from IMPDH-1. In embodiments, the split intein is a C-intein or an N-intein from NrdJ-1. In embodiments, the split intein is a C-intein or an N-intein from MtuRec^{Δ297}. In embodiments, the split intein is from gp41-1. In embodiments, the split intein is a C-intein or an N-intein from AovDnaE. In embodiments, the split intein is a C-intein or an N-intein from AspDnaE. In embodiments, the split intein is a C-intein or an N-intein from AvaDnaE. In embodiments, the split intein is a C-intein or an N-intein from Cra(C5505)DnaE. In embodiments, the split intein is a C-intein or an N-intein from Csp(CCY0110)DnaE. In embodiments, the split intein is a C-intein or an N-intein from CwaDnaE. In embodiments, the split intein is a C-intein or an N-intein from Maer(NIES843)DnaE. In embodiments, the split intein is a C-intein or an N-intein from Mcht(PCC7420)DnaE, MtuRecA^{Δ300}. In embodiments, the split intein is a C-intein or an N-intein from NspDnaE. In embodiments, the split intein is a C-intein or an N-intein from OliDnaE. In embodiments, the split intein is a C-intein or an N-intein from Sel(PC7942)DnaE. In embodiments, the split intein is a C-intein or an N-intein from Ssp(PCC7002)DnaE. In embodiments, the split intein is a C-intein or an N-intein from TerDnaE-3. In embodiments, the split intein is a C-intein or an N-intein from TelDnaE. In embodiments, the split intein is a C-intein or an N-intein from TvuDnaE. In embodiments, the split intein is a C-intein or an N-intein from NeqPol. In embodiments, the split intein is a C-intein or an N-intein from TerThyX^{Δ132}. The split site for each intein is known in the art. See Aranko A S, Wlodawer A, Iwaï H. Nature's recipe for splitting inteins. Protein Eng Des Sel. 2014 August; 27(8):263-71.

In embodiments, the first intein has a length of about 1 to about 30 amino acid residues. In embodiments, the first intein has a length of about 2 to about 30 amino acid residues. In embodiments, the first intein has a length of about 3 to about 30 amino acid residues. In embodiments, the first intein has a length of about 4 to about 30 amino acid residues. In embodiments, the first intein has a length of about 5 to about 30 amino acid residues. In embodiments, the first intein has a length of about 6 to about 30 amino acid residues. In embodiments, the first intein has a length of about 7 to about 30 amino acid residues. In embodiments, the first intein has a length of about 8 to about 30 amino acid residues. In embodiments, the first intein has a length of about 9 to about 30 amino acid residues. In embodiments, the first intein has a length of about 10 to about 30 amino acid residues. In embodiments, the first intein has a length of about 11 to about 30 amino acid residues. In embodiments, the first intein has a length of about 12 to about 30 amino acid residues. In embodiments, the first intein has a length of about 13 to about 30 amino acid residues. In embodiments, the first intein has a length of about 14 to about 30 amino acid residues. In embodiments, the first intein has a length of about 15 to about 30 amino acid residues. In embodiments, the first intein has a length of about 16 to about 30 amino acid residues. In embodiments, the first intein has a length of about 17 to about 30 amino acid residues. In embodiments, the first intein has a length of about 18 to about 30 amino acid residues. In embodiments, the first intein has a length of about 19 to about 30 amino acid residues. In embodiments, the first intein has a length of about 20 to about 30 amino acid residues. In embodiments, the first intein has a length of about 21 to about 30 amino acid residues. In embodiments, the first intein has a length of about 22 to about 30 amino acid residues. In embodiments, the first intein has a length of about 23 to about 30 amino acid residues. In embodiments, the first intein has a length of about 24 to about 30 amino acid residues. In embodiments, the first intein has a length of about 25 to about 30 amino acid residues. In embodiments, the first intein has a length of about 26 to about 30 amino acid residues. In embodiments, the first intein has a length of about 27 to about 30 amino acid residues. In embodiments, the first intein has a length of about 28 to about 30 amino acid residues. In embodiments, the first intein has a length of about 29 to about 30 amino acid residues.

In embodiments, the first intein has a length of about 1 to about 29 amino acid residues. In embodiments, the first intein has a length of about 1 to about 28 amino acid residues. In embodiments, the first intein has a length of about 1 to about 27 amino acid residues. In embodiments, the first intein has a length of about 1 to about 26 amino acid residues. In embodiments, the first intein has a length of about 1 to about 25 amino acid residues. In embodiments, the first intein has a length of about 1 to about 24 amino acid residues. In embodiments, the first intein has a length of about 1 to about 23 amino acid residues. In embodiments, the first intein has a length of about 1 to about 22 amino acid residues. In embodiments, the first intein has a length of about 1 to about 21 amino acid residues. In embodiments, the first intein has a length of about 1 to about 20 amino acid residues. In embodiments, the first intein has a length of about 1 to about 19 amino acid residues. In embodiments, the first intein has a length of about 1 to about 18 amino acid residues. In embodiments, the first intein has a length of about 1 to about 17 amino acid residues. In embodiments, the first intein has a length of about 1 to about 16 amino acid residues. In embodiments, the first intein has a length of about 1 to about 15 amino acid residues. In embodiments, the first intein has a length of about 1 to about 14 amino acid residues. In embodiments, the first intein has a length of about 1 to about 13 amino acid residues. In embodiments, the first intein has a length of about 1 to about 12 amino acid residues. In embodiments, the first intein has a length of about 1 to about 11 amino acid residues. In embodiments, the first intein has a length of about 1 to about 10 amino acid residues. In embodiments, the first intein has a length of about 1 to about 9 amino acid residues. In embodiments, the first intein has a length of about 1 to about 8 amino acid residues. In embodiments, the first intein has a length of about 1 to about 7 amino acid residues. In embodiments, the first intein has a length of about 1 to about 6 amino acid residues. In embodiments, the first intein has a length of about 1 to about 5 amino acid residues. In embodiments, the first intein has a length of about 1 to about 4 amino acid residues. In embodiments, the first intein has a length of about 1 to about 3 amino acid residues. In embodiments, the first intein has a length of about 1 to about 2 amino acid residues. In embodiments, the first intein has a length of 1 to 30 amino acid residues. In embodiments, the first intein has a length of 2 to 30 amino acid residues. In embodiments, the first intein has a length of 3 to 30 amino acid residues. In embodiments, the first intein has a length of 4 to 30 amino acid residues. In embodiments, the first intein has a length of 5 to 30 amino acid residues. In embodiments, the first intein has a length of 6 to 30 amino acid residues. In embodiments, the first intein has a length of 7 to 30 amino acid residues. In embodiments, the first intein has a length of 8 to 30 amino acid residues. In embodiments, the first intein has a length of 9 to 30 amino acid residues. In embodiments, the first intein has a length of 10 to 30 amino acid residues. In embodiments, the first intein has a length of 11 to 30 amino acid residues. In embodiments, the first intein has a length of 12 to 30 amino acid residues. In embodiments, the first intein has a length of 13 to 30 amino acid residues. In embodiments, the first intein has a length of 14 to 30 amino acid residues. In embodiments, the first intein has a length of 15 to 30 amino acid residues. In embodiments, the first intein has a length of 16 to 30 amino acid residues. In embodiments, the first intein has a length of 17 to 30 amino acid residues. In embodiments, the first intein has a length of 18 to 30 amino acid residues. In embodiments, the first intein has a length of 19 to 30 amino acid residues. In embodiments, the first intein has a length of 20 to 30 amino acid residues. In embodiments, the first intein has a length of 21 to 30 amino acid residues. In embodiments, the first intein has a length of 22 to 30 amino acid residues. In embodiments, the first intein has a length of 23 to 30 amino acid residues. In embodiments, the first intein has a length of 24 to 30 amino acid residues. In embodiments, the first intein has a length of 25 to 30 amino acid residues. In embodiments, the first intein has a length of 26 to 30 amino acid residues. In embodiments, the first intein has a length of 27 to 30 amino acid residues. In embodiments, the first intein has a length of 28 to 30 amino acid residues. In embodiments, the first intein has a length of 29 to 30 amino acid residues.

In embodiments, the first intein has a length of 1 to 29 amino acid residues. In embodiments, the first intein has a length of 1 to 28 amino acid residues. In embodiments, the first intein has a length of 1 to 27 amino acid residues. In embodiments, the first intein has a length of 1 to 26 amino acid residues. In embodiments, the first intein has a length of 1 to 25 amino acid residues. In embodiments, the first intein has a length of 1 to 24 amino acid residues. In embodiments, the first intein has a length of 1 to 23 amino acid residues. In embodiments, the first intein has a length of 1 to 22 amino acid residues. In embodiments, the first intein has a length of 1 to 21 amino acid residues. In embodiments, the first intein has a length of 1 to 20 amino acid residues. In embodiments, the first intein has a length of 1 to 19 amino acid residues. In embodiments, the first intein has a length of 1 to 18 amino acid residues. In embodiments, the first intein has a length of 1 to 17 amino acid residues. In embodiments, the first intein has a length of 1 to 16 amino acid residues. In embodiments, the first intein has a length of 1 to 15 amino acid residues. In embodiments, the first intein has a length of 1 to 14 amino acid residues. In embodiments, the first intein has a length of 1 to 13 amino acid residues. In embodiments, the first intein has a length of 1 to 12 amino acid residues. In embodiments, the first intein has a length of 1 to 11 amino acid residues. In embodiments, the first intein has a length of 1 to 10 amino acid residues. In embodiments, the first intein has a length of 1 to 9 amino acid residues. In embodiments, the first intein has a length of 1 to 8 amino acid residues. In embodiments, the first intein has a length of 1 to 7 amino acid residues. In embodiments, the first intein has a length of 1 to 6 amino acid residues. In embodiments, the first intein has a length of 1 to 5 amino acid residues. In embodiments, the first intein has a length of 1 to 4 amino acid residues. In embodiments, the first intein has a length of 1 to 3 amino acid residues. In embodiments, the first intein has a length of 1 to 2 amino acid residues.

In embodiments, the first intein has a length of 1 amino acid residue. In embodiments, the first intein has a length of 2 amino acid residues. In embodiments, the first intein has a length of 3 amino acid residues. In embodiments, the first intein has a length of 4 amino acid residues. In embodiments, the first intein has a length of 5 amino acid residues. In embodiments, the first intein has a length of 6 amino acid residues. In embodiments, the first intein has a length of 7 amino acid residues. In embodiments, the first intein has a length of 8 amino acid residues. In embodiments, the first intein has a length of 9 amino acid residues. In embodiments, the first intein has a length of 10 amino acid residues. In embodiments, the first intein has a length of 11 amino acid residues. In embodiments, the first intein has a length of 12 amino acid residues. In embodiments, the first intein has a length of 13 amino acid residues. In embodiments, the first intein has a length of 14 amino acid residues. In embodiments, the first intein has a length of 15 amino acid residues. In embodiments, the first intein has a length of 16 amino acid residues. In embodiments, the first intein has a length of 17 amino acid residues. In embodiments, the first intein has a length of 18 amino acid residues. In embodiments, the first intein has a length of 19 amino acid residues. In embodiments, the first intein has a length of 20 amino acid residues. In embodiments, the first intein has a length of 21 amino acid residues. In embodiments, the first intein has a length of 22 amino acid residues. In embodiments, the first intein has a length of 23 amino acid residues. In embodiments, the first intein has a length of 24 amino acid residues. In embodiments, the first intein has a length of 25 amino acid residues. In embodiments, the first intein has a length of 26 amino acid residues. In embodiments, the first intein has a length of 27 amino acid residues. In embodiments, the first intein has a length of 28 amino acid residues. In embodiments, the first intein has a length of 29 amino acid residues. In embodiments, the first intein has a length of 30 amino acid residues.

In embodiments, the first intein has a length of about 1 to about 300 amino acid residues. In embodiments, the first intein has a length of about 10 to about 300 amino acid residues. In embodiments, the first intein has a length of about 20 to about 300 amino acid residues. In embodiments, the first intein has a length of about 30 to about 300 amino acid residues. In embodiments, the first intein has a length of about 40 to about 300 amino acid residues. In embodiments, the first intein has a length of about 50 to about 300 amino acid residues. In embodiments, the first intein has a length of about 60 to about 300 amino acid residues. In embodiments, the first intein has a length of about 70 to about 300 amino acid residues. In embodiments, the first intein has a length of about 80 to about 300 amino acid residues. In embodiments, the first intein has a length of about 90 to about 300 amino acid residues. In embodiments, the first intein has a length of about 100 to about 300 amino acid residues. In embodiments, the first intein has a length of about 110 to about 300 amino acid residues. In embodiments, the first intein has a length of about 120 to about 300 amino acid residues. In embodiments, the first intein has a length of about 130 to about 300 amino acid residues. In embodiments, the first intein has a length of about 140 to about 300 amino acid residues. In embodiments, the first intein has a length of about 150 to about 300 amino acid residues. In embodiments, the first intein has a length of about 160 to about 300 amino acid residues. In embodiments, the first intein has a length of about 170 to about 300 amino acid residues. In embodiments, the first intein has a length of about 180 to about 300 amino acid residues. In embodiments, the first intein has a length of about 190 to about 300 amino acid residues. In embodiments, the first intein has a length of about 200 to about 300 amino acid residues. In embodiments, the first intein has a length of about 210 to about 300 amino acid residues. In embodiments, the first intein has a length of about 220 to about 300 amino acid residues. In embodiments, the first intein has a length of about 230 to about 300 amino acid residues. In embodiments, the first intein has a length of about 240 to about 300 amino acid residues. In embodiments, the first intein has a length of about 250 to about 300 amino acid residues. In embodiments, the first intein has a length of about 260 to about 300 amino acid residues. In embodiments, the first intein has a length of about 270 to about 300 amino acid residues. In embodiments, the first intein has a length of about 280 to about 300 amino acid residues. In embodiments, the first intein has a length of about 290 to about 300 amino acid residues.

In embodiments, the first intein has a length of about 1 to about 290 amino acid residues. In embodiments, the first intein has a length of about 1 to about 280 amino acid residues. In embodiments, the first intein has a length of about 1 to about 270 amino acid residues. In embodiments, the first intein has a length of about 1 to about 260 amino acid residues. In embodiments, the first intein has a length of about 1 to about 250 amino acid residues. In embodiments, the first intein has a length of about 1 to about 240 amino acid residues. In embodiments, the first intein has a length of about 1 to about 230 amino acid residues. In embodiments, the first intein has a length of about 1 to about 220 amino acid residues. In embodiments, the first intein has a length of about 1 to about 210 amino acid residues. In embodiments, the first intein has a length of about 1 to about 200 amino acid residues. In embodiments, the first intein has a length of about 1 to about 190 amino acid residues. In embodiments, the first intein has a length of about 1 to about 180 amino acid residues. In embodiments, the first intein has a length of about 1 to about 170 amino acid residues. In embodiments, the first intein has a length of about 1 to about 160 amino acid residues. In embodiments, the first intein has a length of about 1 to about 150 amino acid residues. In embodiments, the first intein has a length of about 1 to about 140 amino acid residues. In embodiments, the first intein has a length of about 1 to about 130 amino acid residues. In embodiments, the first intein has a length of about 1 to about 120 amino acid residues. In embodiments, the first intein has a length of about 1 to about 110 amino acid residues. In embodiments, the first intein has a length of about 1 to about 100 amino acid residues. In embodiments, the first intein has a length of about 1 to about 90 amino acid residues. In embodiments, the first intein has a length of about 1 to about 80 amino acid residues. In embodiments, the first intein has a length of about 1 to about 70 amino acid residues. In embodiments, the first intein has a length of about 1 to about 60 amino acid residues. In embodiments, the first intein has a length of about 1 to about 50 amino acid residues. In embodiments, the first intein has a length of about 1 to about 40 amino acid residues. In embodiments, the first intein has a length of about 1 to about 30 amino acid residues. In embodiments, the first intein has a length of about 1 to about 20 amino acid residues. In embodiments, the first intein has a length of about 1 to about 10 amino acid residues.

In embodiments, the first intein has a length of 1 to 300 amino acid residues. In embodiments, the first intein has a length of 10 to 300 amino acid residues. In embodiments, the first intein has a length of 20 to 300 amino acid residues. In embodiments, the first intein has a length of 30 to 300 amino acid residues. In embodiments, the first intein has a length of 40 to 300 amino acid residues. In embodiments, the first intein has a length of 50 to 300 amino acid residues. In embodiments, the first intein has a length of 60 to 300 amino acid residues. In embodiments, the first intein has a length of 70 to 300 amino acid residues. In embodiments, the first intein has a length of 80 to 300 amino acid residues. In embodiments, the first intein has a length of 90 to 300 amino acid residues. In embodiments, the first intein has a length of 100 to 300 amino acid residues. In embodiments, the first intein has a length of 110 to 300 amino acid residues. In embodiments, the first intein has a length of 120 to 300 amino acid residues. In embodiments, the first intein has a length of 130 to 300 amino acid residues. In embodiments, the first intein has a length of 140 to 300 amino acid residues. In embodiments, the first intein has a length of 150 to 300 amino acid residues. In embodiments, the first intein has a length of 160 to 300 amino acid residues. In embodiments, the first intein has a length of 170 to 300 amino acid residues. In embodiments, the first intein has a length of 180 to 300 amino acid residues. In embodiments, the first intein has a length of 190 to 300 amino acid residues. In embodiments, the first intein has a length of 200 to 300 amino acid residues. In embodiments, the first intein has a length of 210 to 300 amino acid residues. In embodiments, the first intein has a length of 220 to 300 amino acid residues. In embodiments, the first intein has a length of 230 to 300 amino acid residues. In embodiments, the first intein has a length of 240 to 300 amino acid residues. In embodiments, the first intein has a length of 250 to 300 amino acid residues. In embodiments, the first intein has a length of 260 to 300 amino acid residues. In embodiments, the first intein has a length of 270 to 300 amino acid residues. In embodiments, the first intein has a length of 280 to 300 amino acid residues. In embodiments, the first intein has a length of 290 to 300 amino acid residues.

In embodiments, the first intein has a length of 1 to 30 amino acid residues. In embodiments, the first intein has a length of 2 to 30 amino acid residues. In embodiments, the first intein has a length of 3 to 30 amino acid residues. In embodiments, the first intein has a length of 4 to 30 amino acid residues. In embodiments, the first intein has a length of 5 to 30 amino acid residues. In embodiments, the first intein has a length of 6 to 30 amino acid residues. In embodiments, the first intein has a length of 7 to 30 amino acid residues. In embodiments, the first intein has a length of 8 to 30 amino acid residues. In embodiments, the first intein has a length of 9 to 30 amino acid residues. In embodiments, the first intein has a length of 10 to 30 amino acid residues. In embodiments, the first intein has a length of 11 to 30 amino acid residues. In embodiments, the first intein has a length of 12 to 30 amino acid residues. In embodiments, the first intein has a length of 13 to 30 amino acid residues. In embodiments, the first intein has a length of 14 to 30 amino acid residues. In embodiments, the first intein has a length of 15 to 30 amino acid residues. In embodiments, the first intein has a length of 16 to 30 amino acid residues. In embodiments, the first intein has a length of 17 to 30 amino acid residues. In embodiments, the first intein has a length of 18 to 30 amino acid residues. In embodiments, the first intein has a length of 19 to 30 amino acid residues. In embodiments, the first intein has a length of 20 to 30 amino acid residues. In embodiments, the first intein has a length of 21 to 30 amino acid residues. In embodiments, the first intein has a length of 22 to 30 amino acid residues. In embodiments, the first intein has a length of 23 to 30 amino acid residues. In embodiments, the first intein has a length of 24 to 30 amino acid residues. In embodiments, the first intein has a length of 25 to 30 amino acid residues. In embodiments, the first intein has a length of 26 to 30 amino acid residues. In embodiments, the first intein has a length of 27 to 30 amino acid residues. In embodiments, the first intein has a length of 28 to 30 amino acid residues. In embodiments, the first intein has a length of 29 to 30 amino acid residues.

In embodiments, the first intein has a length of 1 to 29 amino acid residues. In embodiments, the first intein has a length of 1 to 28 amino acid residues. In embodiments, the first intein has a length of 1 to 27 amino acid residues. In embodiments, the first intein has a length of 1 to 26 amino acid residues. In embodiments, the first intein has a length of 1 to 25 amino acid residues. In embodiments, the first intein has a length of 1 to 24 amino acid residues. In embodiments, the first intein has a length of 1 to 23 amino acid residues. In embodiments, the first intein has a length of 1 to 22 amino acid residues. In embodiments, the first intein has a length of 1 to 21 amino acid residues. In embodiments, the first intein has a length of 1 to 20 amino acid residues. In embodiments, the first intein has a length of 1 to 19 amino acid residues. In embodiments, the first intein has a length of 1 to 18 amino acid residues. In embodiments, the first intein has a length of 1 to 17 amino acid residues. In embodiments, the first intein has a length of 1 to 16 amino acid residues. In embodiments, the first intein has a length of 1 to 15 amino acid residues. In embodiments, the first intein has a length of 1 to 14 amino acid residues. In embodiments, the first intein has a length of 1 to 13 amino acid residues. In embodiments, the first intein has a length of 1 to 12 amino acid residues. In embodiments, the first intein has a length of 1 to 11 amino acid residues. In embodiments, the first intein has a length of 1 to 10 amino acid residues. In embodiments, the first intein has a length of 1 to 9 amino acid residues. In embodiments, the first intein has a length of 1 to 8 amino acid residues. In embodiments, the first intein has a length of 1 to 7 amino acid residues. In embodiments, the first intein has a length of 1 to 6 amino acid residues. In embodiments, the first intein has a length of 1 to 5 amino acid residues. In embodiments, the first intein has a length of 1 to 4 amino acid residues. In embodiments, the first intein has a length of 1 to 3 amino acid residues. In embodiments, the first intein has a length of 1 to 2 amino acid residues.

In embodiments, the first intein has a length of 1 amino acid residue. In embodiments, the first intein has a length of 2 amino acid residues. In embodiments, the first intein has a length of 3 amino acid residues. In embodiments, the first intein has a length of 4 amino acid residues. In embodiments, the first intein has a length of 5 amino acid residues. In embodiments, the first intein has a length of 6 amino acid residues. In embodiments, the first intein has a length of 7 amino acid residues. In embodiments, the first intein has a length of 8 amino acid residues. In embodiments, the first intein has a length of 9 amino acid residues. In embodiments, the first intein has a length of 10 amino acid residues. In embodiments, the first intein has a length of 11 amino acid residues. In embodiments, the first intein has a length of 12 amino acid residues. In embodiments, the first intein has a length of 13 amino acid residues. In embodiments, the first intein has a length of 14 amino acid residues. In embodiments, the first intein has a length of 15 amino acid residues. In embodiments, the first intein has a length of 16 amino acid residues. In embodiments, the first intein has a length of 17 amino acid residues. In embodiments, the first intein has a length of 18 amino acid residues. In embodiments, the first intein has a length of 19 amino acid residues. In embodiments, the first intein has a length of 20 amino acid residues. In embodiments, the first intein has a length of 21 amino acid residues. In embodiments, the first intein has a length of 22 amino acid residues. In embodiments, the first intein has a length of 23 amino acid residues. In embodiments, the first intein has a length of 24 amino acid residues. In embodiments, the first intein has a length of 25 amino acid residues. In embodiments, the first intein has a length of 26 amino acid residues. In embodiments, the first intein has a length of 27 amino acid residues. In embodiments, the first intein has a length of 28 amino acid residues. In embodiments, the first intein has a length of 29 amino acid residues. In embodiments, the first intein has a length of 30 amino acid residues.

In embodiments, the transmembrane domain is a PD-1 transmembrane domain, a PD-L1 transmembrane domain, an EGFR transmembrane domain, a proteorhodopsin transmembrane domain, a receptor tyrosine kinase transmembrane domain, a notch receptor transmembrane domain, a hemagglutinin transmembrane domain, a neuraminidase transmembrane domain, an ACE-2 transmembrane domain, a rhomboid protease transmembrane domain, or a WALP peptide. In embodiments, the transmembrane domain is a PD-1 transmembrane domain. In embodiments, the transmembrane domain is a PD-L1 transmembrane domain. In embodiments, the transmembrane domain is an EGFR transmembrane domain. In embodiments, the transmembrane domain is a proteorhodopsin transmembrane domain. In embodiments, the transmembrane domain is a receptor tyrosine kinase transmembrane domain. In embodiments, the transmembrane domain is a notch receptor transmembrane domain. In embodiments, the transmembrane domain is a hemagglutinin transmembrane domain. In embodiments, the transmembrane domain is a neuraminidase transmembrane domain. In embodiments, the transmembrane domain is an ACE-2 transmembrane domain. In embodiments, the transmembrane domain is a rhomboid protease transmembrane domain. In embodiments, the transmembrane domain is a WALP peptide. In embodiments, further including a second polypeptide covalently bound to the first intein. In further embodiments, the second polypeptide is covalently bound to a second intein of the split intein pair. In embodiments, the first intein is a C-intein and the second intein is an N-intein. In embodiments, the first intein is an N-intein and the second intein is a C-intein. In embodiments, the amino acid length of the first intein is shorter than the amino acid length of the second intein.

In embodiments, the second intein has a length of about 1 to about 300 amino acid residues. In embodiments, the second intein has a length of about 5 to about 300 amino acid residues. In embodiments, the second intein has a length of about 10 to about 300 amino acid residues. In embodiments, the second intein has a length of about 20 to about 300 amino acid residues. In embodiments, the second intein has a length of about 30 to about 300 amino acid residues. In embodiments, the second intein has a length of about 40 to about 300 amino acid residues. In embodiments, the second intein has a length of about 50 to about 300 amino acid residues. In embodiments, the second intein has a length of about 60 to about 300 amino acid residues. In embodiments, the second intein has a length of about 70 to about 300 amino acid residues. In embodiments, the second intein has a length of about 80 to about 300 amino acid residues. In embodiments, the second intein has a length of about 90 to about 300 amino acid residues. In embodiments, the second intein has a length of about 100 to about 300 amino acid residues. In embodiments, the second intein has a length of about 110 to about 300 amino acid residues. In embodiments, the second intein has a length of about 120 to about 300 amino acid residues. In embodiments, the second intein has a length of about 130 to about 300 amino acid residues. In embodiments, the second intein has a length of about 140 to about 300 amino acid residues. In embodiments, the second intein has a length of about 150 to about 300 amino acid residues. In embodiments, the second intein has a length of about 160 to about 300 amino acid residues. In embodiments, the second intein has a length of about 170 to about 300 amino acid residues. In embodiments, the second intein has a length of about 180 to about 300 amino acid residues. In embodiments, the second intein has a length of about 190 to about 300 amino acid residues. In embodiments, the second intein has a length of about 200 to about 300 amino acid residues. In embodiments, the second intein has a length of about 210 to about 300 amino acid residues. In embodiments, the second intein has a length of about 220 to about 300 amino acid residues. In embodiments, the second intein has a length of about 230 to about 300 amino acid residues. In embodiments, the second intein has a length of about 240 to about 300 amino acid residues. In embodiments, the second intein has a length of about 250 to about 300 amino acid residues. In embodiments, the second intein has a length of about 260 to about 300 amino acid residues. In embodiments, the second intein has a length of about 270 to about 300 amino acid residues. In embodiments, the second intein has a length of about 280 to about 300 amino acid residues. In embodiments, the second intein has a length of about 290 to about 300 amino acid residues.

In embodiments, the second intein has a length of about 1 to about 290 amino acid residues. In embodiments, the second intein has a length of about 1 to about 280 amino acid residues. In embodiments, the second intein has a length of about 1 to about 270 amino acid residues. In embodiments, the second intein has a length of about 1 to about 260 amino acid residues. In embodiments, the second intein has a length of about 1 to about 250 amino acid residues. In embodiments, the second intein has a length of about 1 to about 240 amino acid residues. In embodiments, the second intein has a length of about 1 to about 230 amino acid residues. In embodiments, the second intein has a length of about 1 to about 220 amino acid residues. In embodiments, the second intein has a length of about 1 to about 210 amino acid residues. In embodiments, the second intein has a length of about 1 to about 200 amino acid residues. In embodiments, the second intein has a length of about 1 to about 190 amino acid residues. In embodiments, the second intein has a length of about 1 to about 180 amino acid residues. In embodiments, the second intein has a length of about 1 to about 170 amino acid residues. In embodiments, the second intein has a length of about 1 to about 160 amino acid residues. In embodiments, the second intein has a length of about 1 to about 150 amino acid residues. In embodiments, the second intein has a length of about 1 to about 140 amino acid residues. In embodiments, the second intein has a length of about 1 to about 130 amino acid residues. In embodiments, the second intein has a length of about 1 to about 120 amino acid residues. In embodiments, the second intein has a length of about 1 to about 110 amino acid residues. In embodiments, the second intein has a length of about 1 to about 100 amino acid residues. In embodiments, the second intein has a length of about 1 to about 90 amino acid residues. In embodiments, the second intein has a length of about 1 to about 80 amino acid residues. In embodiments, the second intein has a length of about 1 to about 70 amino acid residues. In embodiments, the second intein has a length of about 1 to about 60 amino acid residues. In embodiments, the second intein has a length of about 1 to about 50 amino acid residues. In embodiments, the second intein has a length of about 1 to about 40 amino acid residues. In embodiments, the second intein has a length of about 1 to about 30 amino acid residues. In embodiments, the second intein has a length of about 1 to about 20 amino acid residues. In embodiments, the second intein has a length of about 1 to about 10 amino acid residues. In embodiments, the second intein has a length of about 1 to about 5 amino acid residues.

In embodiments, the second intein has a length of 1 to 300 amino acid residues. In embodiments, the second intein has a length of 5 to 300 amino acid residues. In embodiments, the second intein has a length of 10 to 300 amino acid residues. In embodiments, the second intein has a length of 20 to 300 amino acid residues. In embodiments, the second intein has a length of 30 to 300 amino acid residues. In embodiments, the second intein has a length of 40 to 300 amino acid residues. In embodiments, the second intein has a length of 50 to 300 amino acid residues. In embodiments, the second intein has a length of 60 to 300 amino acid residues. In embodiments, the second intein has a length of 70 to 300 amino acid residues. In embodiments, the second intein has a length of 80 to 300 amino acid residues. In embodiments, the second intein has a length of 90 to 300 amino acid residues. In embodiments, the second intein has a length of 100 to 300 amino acid residues. In embodiments, the second intein has a length of 110 to 300 amino acid residues. In embodiments, the second intein has a length of 120 to 300 amino acid residues. In embodiments, the second intein has a length of 130 to 300 amino acid residues. In embodiments, the second intein has a length of 140 to 300 amino acid residues. In embodiments, the second intein has a length of 150 to 300 amino acid residues. In embodiments, the second intein has a length of 160 to 300 amino acid residues. In embodiments, the second intein has a length of 170 to 300 amino acid residues. In embodiments, the second intein has a length of 180 to 300 amino acid residues. In embodiments, the second intein has a length of 190 to 300 amino acid residues. In embodiments, the second intein has a length of 200 to 300 amino acid residues. In embodiments, the second intein has a length of 210 to 300 amino acid residues. In embodiments, the second intein has a length of 220 to 300 amino acid residues. In embodiments, the second intein has a length of 230 to 300 amino acid residues. In embodiments, the second intein has a length of 240 to 300 amino acid residues. In embodiments, the second intein has a length of 250 to 300 amino acid residues. In embodiments, the second intein has a length of 260 to 300 amino acid residues. In embodiments, the second intein has a length of 270 to 300 amino acid residues. In embodiments, the second intein has a length of 280 to 300 amino acid residues. In embodiments, the second intein has a length of 290 to 300 amino acid residues.

In embodiments, the second intein has a length of 1 to 290 amino acid residues. In embodiments, the second intein has a length of 1 to 280 amino acid residues. In embodiments, the second intein has a length of 1 to 270 amino acid residues. In embodiments, the second intein has a length of 1 to 260 amino acid residues. In embodiments, the second intein has a length of 1 to 250 amino acid residues. In embodiments, the second intein has a length of 1 to 240 amino acid residues. In embodiments, the second intein has a length of 1 to 230 amino acid residues. In embodiments, the second intein has a length of 1 to 220 amino acid residues. In embodiments, the second intein has a length of 1 to 210 amino acid residues. In embodiments, the second intein has a length of 1 to 200 amino acid residues. In embodiments, the second intein has a length of 1 to 190 amino acid residues. In embodiments, the second intein has a length of 1 to 180 amino acid residues. In embodiments, the second intein has a length of 1 to 170 amino acid residues. In embodiments, the second intein has a length of 1 to 160 amino acid residues. In embodiments, the second intein has a length of 1 to 150 amino acid residues. In embodiments, the second intein has a length of 1 to 140 amino acid residues. In embodiments, the second intein has a length of 1 to 130 amino acid residues. In embodiments, the second intein has a length of 1 to 120 amino acid residues. In embodiments, the second intein has a length of 1 to 110 amino acid residues. In embodiments, the second intein has a length of 1 to 100 amino acid residues. In embodiments, the second intein has a length of 1 to 90 amino acid residues. In embodiments, the second intein has a length of 1 to 80 amino acid residues. In embodiments, the second intein has a length of 1 to 70 amino acid residues. In embodiments, the second intein has a length of 1 to 60 amino acid residues. In embodiments, the second intein has a length of 1 to 50 amino acid residues. In embodiments, the second intein has a length of 1 to 40 amino acid residues. In embodiments, the second intein has a length of 1 to 30 amino acid residues. In embodiments, the second intein has a length of 1 to 20 amino acid residues. In embodiments, the second intein has a length of 1 to 10 amino acid residues. In embodiments, the second intein has a length of 1 to 5 amino acid residues.

In embodiments, the second polypeptide is an extracellular or intracellular domain of a signaling, receptor, channel, transport, or G-protein coupled receptor (GPCR) membrane protein. In embodiments, the second polypeptide is an extracellular or intracellular domain of a signaling membrane protein. In embodiments, the second polypeptide is an extracellular domain of a signaling membrane protein. In embodiments, the second polypeptide is an intracellular domain of a signaling membrane protein. In embodiments, the second polypeptide is an extracellular or intracellular domain of a receptor membrane protein. In embodiments, the second polypeptide is an extracellular domain of a receptor membrane protein. In embodiments, the second polypeptide is an extracellular domain of a receptor membrane protein. In embodiments, the second polypeptide is an extracellular or intracellular domain of a channel membrane protein. In embodiments, the second polypeptide is an extracellular domain of a channel membrane protein. In embodiments, the second polypeptide is an intracellular domain of a channel membrane protein. In embodiments, the second polypeptide is an extracellular or intracellular domain of a transport membrane protein. In embodiments, the second polypeptide is an extracellular domain of a transport membrane protein. In embodiments, the second polypeptide is an intracellular domain of a transport membrane protein. In embodiments, the second polypeptide is an extracellular or intracellular domain of a G-protein coupled receptor (GPCR) membrane protein. In embodiments, the second polypeptide is an extracellular domain of a G-protein coupled receptor (GPCR) membrane protein. In embodiments, the second polypeptide is an intracellular domain of a G-protein coupled receptor (GPCR) membrane protein.

In embodiments, the extracellular domain is a PD-1 extracellular domain, a PD-L1 extracellular domain, an EGFR extracellular domain, a proteorhodopsin extracellular domain, a receptor tyrosine kinase extracellular domain, a notch receptor extracellular domain, a hemagglutinin extracellular domain, a neuraminidase extracellular domain, an ACE-2 extracellular domain, or a rhomboid protease extracellular domain. In embodiments, the extracellular domain is a PD-1 extracellular domain. In embodiments, the extracellular domain is a PD-L1 extracellular domain. In embodiments, the extracellular domain is an EGFR extracellular domain. In embodiments, the extracellular domain is a proteorhodopsin extracellular domain. In embodiments, the extracellular domain is a receptor tyrosine kinase extracellular domain. In embodiments, the extracellular domain is a notch receptor extracellular domain. In embodiments, the extracellular domain is a hemagglutinin extracellular domain. In embodiments, the extracellular domain is a neuraminidase extracellular domain. In embodiments, the extracellular domain is an ACE-2 extracellular domain. In embodiments, the extracellular domain is a rhomboid protease extracellular domain.

In embodiments, the extracellular domain has a length of about 10 to about 1000 amino acid residues. In embodiments, the extracellular domain has a length of about 20 to about 1000 amino acid residues. In embodiments, the extracellular domain has a length of about 30 to about 1000 amino acid residues. In embodiments, the extracellular domain has a length of about 40 to about 1000 amino acid residues. In embodiments, the extracellular domain has a length of about 50 to about 1000 amino acid residues. In embodiments, the extracellular domain has a length of about 60 to about 1000 amino acid residues. In embodiments, the extracellular domain has a length of about 70 to about 1000 amino acid residues. In embodiments, the extracellular domain has a length of about 80 to about 1000 amino acid residues. In embodiments, the extracellular domain has a length of about 90 to about 1000 amino acid residues. In embodiments, the extracellular domain has a length of about 100 to about 1000 amino acid residues. In embodiments, the extracellular domain has a length of about 150 to about 1000 amino acid residues. In embodiments, the extracellular domain has a length of about 200 to about 1000 amino acid residues. In embodiments, the extracellular domain has a length of about 250 to about 1000 amino acid residues. In embodiments, the extracellular domain has a length of about 300 to about 1000 amino acid residues. In embodiments, the extracellular domain has a length of about 350 to about 1000 amino acid residues. In embodiments, the extracellular domain has a length of about 400 to about 1000 amino acid residues. In embodiments, the extracellular domain has a length of about 450 to about 1000 amino acid residues. In embodiments, the extracellular domain has a length of about 500 to about 1000 amino acid residues. In embodiments, the extracellular domain has a length of about 550 to about 1000 amino acid residues. In embodiments, the extracellular domain has a length of about 600 to about 1000 amino acid residues. In embodiments, the extracellular domain has a length of about 650 to about 1000 amino acid residues. In embodiments, the extracellular domain has a length of about 700 to about 1000 amino acid residues. In embodiments, the extracellular domain has a length of about 750 to about 1000 amino acid residues. In embodiments, the extracellular domain has a length of about 800 to about 1000 amino acid residues. In embodiments, the extracellular domain has a length of about 850 to about 1000 amino acid residues. In embodiments, the extracellular domain has a length of about 900 to about 1000 amino acid residues. In embodiments, the extracellular domain has a length of about 950 to about 1000 amino acid residues.

In embodiments, the extracellular domain has a length of about 10 to about 950 amino acid residues. In embodiments, the extracellular domain has a length of about 10 to about 900 amino acid residues. In embodiments, the extracellular domain has a length of about 10 to about 850 amino acid residues. In embodiments, the extracellular domain has a length of about 10 to about 800 amino acid residues. In embodiments, the extracellular domain has a length of about 10 to about 750 amino acid residues. In embodiments, the extracellular domain has a length of about 10 to about 700 amino acid residues. In embodiments, the extracellular domain has a length of about 10 to about 650 amino acid residues. In embodiments, the extracellular domain has a length of about 10 to about 600 amino acid residues. In embodiments, the extracellular domain has a length of about 10 to about 550 amino acid residues. In embodiments, the extracellular domain has a length of about 10 to about 500 amino acid residues. In embodiments, the extracellular domain has a length of about 10 to about 450 amino acid residues. In embodiments, the extracellular domain has a length of about 10 to about 400 amino acid residues. In embodiments, the extracellular domain has a length of about 10 to about 350 amino acid residues. In embodiments, the extracellular domain has a length of about 10 to about 300 amino acid residues. In embodiments, the extracellular domain has a length of about 10 to about 250 amino acid residues. In embodiments, the extracellular domain has a length of about 10 to about 200 amino acid residues. In embodiments, the extracellular domain has a length of about 10 to about 150 amino acid residues. In embodiments, the extracellular domain has a length of about 10 to about 100 amino acid residues. In embodiments, the extracellular domain has a length of about 10 to about 90 amino acid residues. In embodiments, the extracellular domain has a length of about 10 to about 80 amino acid residues. In embodiments, the extracellular domain has a length of about 10 to about 70 amino acid residues. In embodiments, the extracellular domain has a length of about 10 to about 60 amino acid residues. In embodiments, the extracellular domain has a length of about 10 to about 50 amino acid residues. In embodiments, the extracellular domain has a length of about 10 to about 40 amino acid residues. In embodiments, the extracellular domain has a length of about 10 to about 30 amino acid residues. In embodiments, the extracellular domain has a length of about 10 to about 20 amino acid residues.

In embodiments, the extracellular domain has a length of 10 to 1000 amino acid residues. In embodiments, the extracellular domain has a length of 20 to 1000 amino acid residues. In embodiments, the extracellular domain has a length of 30 to 1000 amino acid residues. In embodiments, the extracellular domain has a length of 40 to 1000 amino acid residues. In embodiments, the extracellular domain has a length of 50 to 1000 amino acid residues. In embodiments, the extracellular domain has a length of 60 to 1000 amino acid residues. In embodiments, the extracellular domain has a length of 70 to 1000 amino acid residues. In embodiments, the extracellular domain has a length of 80 to 1000 amino acid residues. In embodiments, the extracellular domain has a length of 90 to 1000 amino acid residues. In embodiments, the extracellular domain has a length of 100 to 1000 amino acid residues. In embodiments, the extracellular domain has a length of 150 to 1000 amino acid residues. In embodiments, the extracellular domain has a length of 200 to 1000 amino acid residues. In embodiments, the extracellular domain has a length of 250 to 1000 amino acid residues. In embodiments, the extracellular domain has a length of 300 to 1000 amino acid residues. In embodiments, the extracellular domain has a length of 350 to 1000 amino acid residues. In embodiments, the extracellular domain has a length of 400 to 1000 amino acid residues. In embodiments, the extracellular domain has a length of 450 to 1000 amino acid residues. In embodiments, the extracellular domain has a length of 500 to 1000 amino acid residues. In embodiments, the extracellular domain has a length of 550 to 1000 amino acid residues. In embodiments, the extracellular domain has a length of 600 to 1000 amino acid residues. In embodiments, the extracellular domain has a length of 650 to 1000 amino acid residues. In embodiments, the extracellular domain has a length of 700 to 1000 amino acid residues. In embodiments, the extracellular domain has a length of 750 to 1000 amino acid residues. In embodiments, the extracellular domain has a length of 800 to 1000 amino acid residues. In embodiments, the extracellular domain has a length of 850 to 1000 amino acid residues. In embodiments, the extracellular domain has a length of 900 to 1000 amino acid residues. In embodiments, the extracellular domain has a length of 950 to 1000 amino acid residues.

In embodiments, the extracellular domain has a length of 10 to 950 amino acid residues. In embodiments, the extracellular domain has a length of 10 to 900 amino acid residues. In embodiments, the extracellular domain has a length of 10 to 850 amino acid residues. In embodiments, the extracellular domain has a length of 10 to 800 amino acid residues. In embodiments, the extracellular domain has a length of 10 to 750 amino acid residues. In embodiments, the extracellular domain has a length of 10 to 700 amino acid residues. In embodiments, the extracellular domain has a length of 10 to 650 amino acid residues. In embodiments, the extracellular domain has a length of 10 to 600 amino acid residues. In embodiments, the extracellular domain has a length of 10 to 550 amino acid residues. In embodiments, the extracellular domain has a length of 10 to 500 amino acid residues. In embodiments, the extracellular domain has a length of 10 to 450 amino acid residues. In embodiments, the extracellular domain has a length of 10 to 400 amino acid residues. In embodiments, the extracellular domain has a length of 10 to 350 amino acid residues. In embodiments, the extracellular domain has a length of 10 to 300 amino acid residues. In embodiments, the extracellular domain has a length of 10 to 250 amino acid residues. In embodiments, the extracellular domain has a length of 10 to 200 amino acid residues. In embodiments, the extracellular domain has a length of 10 to 150 amino acid residues. In embodiments, the extracellular domain has a length of 10 to 100 amino acid residues. In embodiments, the extracellular domain has a length of 10 to 90 amino acid residues. In embodiments, the extracellular domain has a length of 10 to 80 amino acid residues. In embodiments, the extracellular domain has a length of 10 to 70 amino acid residues. In embodiments, the extracellular domain has a length of 10 to 60 amino acid residues. In embodiments, the extracellular domain has a length of 10 to 50 amino acid residues. In embodiments, the extracellular domain has a length of 10 to 40 amino acid residues. In embodiments, the extracellular domain has a length of 10 to 30 amino acid residues. In embodiments, the extracellular domain has a length of 10 to 20 amino acid residues.

In embodiments, the intracellular domain is a PD-1 intracellular domain, a PD-L1 intracellular domain, an EGFR intracellular domain, a proteorhodopsin intracellular domain, a receptor tyrosine kinase intracellular domain, a notch receptor intracellular domain, a hemagglutinin intracellular domain, a neuraminidase intracellular domain, an ACE-2 intracellular domain, or a rhomboid protease intracellular domain. In embodiments, the intracellular domain is a PD-1 intracellular domain. In embodiments, the intracellular domain is a PD-L1 intracellular domain. In embodiments, the intracellular domain is an EGFR intracellular domain. In embodiments, the intracellular domain is a proteorhodopsin intracellular domain. In embodiments, the intracellular domain is a receptor tyrosine kinase intracellular domain. In embodiments, the intracellular domain is a notch receptor intracellular domain. In embodiments, the intracellular domain is a hemagglutinin intracellular domain. In embodiments, the intracellular domain is a neuraminidase intracellular domain. In embodiments, the intracellular domain is an ACE-2 intracellular domain. In embodiments, the intracellular domain is a rhomboid protease intracellular domain.

In embodiments, the intracellular domain has a length of about 10 to about 700 amino acid residues. In embodiments, the intracellular domain has a length of about 20 to about 700 amino acid residues. In embodiments, the intracellular domain has a length of about 30 to about 700 amino acid residues. In embodiments, the intracellular domain has a length of about 40 to about 700 amino acid residues. In embodiments, the intracellular domain has a length of about 50 to about 700 amino acid residues. In embodiments, the intracellular domain has a length of about 60 to about 700 amino acid residues. In embodiments, the intracellular domain has a length of about 70 to about 700 amino acid residues. In embodiments, the intracellular domain has a length of about 80 to about 700 amino acid residues. In embodiments, the intracellular domain has a length of about 90 to about 700 amino acid residues. In embodiments, the intracellular domain has a length of about 100 to about 700 amino acid residues. In embodiments, the intracellular domain has a length of about 150 to about 700 amino acid residues. In embodiments, the intracellular domain has a length of about 200 to about 700 amino acid residues. In embodiments, the intracellular domain has a length of about 250 to about 700 amino acid residues. In embodiments, the intracellular domain has a length of about 300 to about 700 amino acid residues. In embodiments, the intracellular domain has a length of about 350 to about 700 amino acid residues. In embodiments, the intracellular domain has a length of about 400 to about 700 amino acid residues. In embodiments, the intracellular domain has a length of about 450 to about 700 amino acid residues. In embodiments, the intracellular domain has a length of about 500 to about 700 amino acid residues. In embodiments, the intracellular domain has a length of about 550 to about 700 amino acid residues. In embodiments, the intracellular domain has a length of about 600 to about 700 amino acid residues. In embodiments, the intracellular domain has a length of about 650 to about 700 amino acid residues.

In embodiments, the intracellular domain has a length of about 10 to about 650 amino acid residues. In embodiments, the intracellular domain has a length of about 10 to about 600 amino acid residues. In embodiments, the intracellular domain has a length of about 10 to about 550 amino acid residues. In embodiments, the intracellular domain has a length of about 10 to about 500 amino acid residues. In embodiments, the intracellular domain has a length of about 10 to about 450 amino acid residues. In embodiments, the intracellular domain has a length of about 10 to about 400 amino acid residues. In embodiments, the intracellular domain has a length of about 10 to about 350 amino acid residues. In embodiments, the intracellular domain has a length of about 10 to about 300 amino acid residues. In embodiments, the intracellular domain has a length of about 10 to about 250 amino acid residues. In embodiments, the intracellular domain has a length of about 10 to about 200 amino acid residues. In embodiments, the intracellular domain has a length of about 10 to about 150 amino acid residues. In embodiments, the intracellular domain has a length of about 10 to about 100 amino acid residues. In embodiments, the intracellular domain has a length of about 10 to about 90 amino acid residues. In embodiments, the intracellular domain has a length of about 10 to about 80 amino acid residues. In embodiments, the intracellular domain has a length of about 10 to about 70 amino acid residues. In embodiments, the intracellular domain has a length of about 10 to about 60 amino acid residues. In embodiments, the intracellular domain has a length of about 10 to about 50 amino acid residues. In embodiments, the intracellular domain has a length of about 10 to about 40 amino acid residues. In embodiments, the intracellular domain has a length of about 10 to about 30 amino acid residues. In embodiments, the intracellular domain has a length of about 10 to about 20 amino acid residues.

In embodiments, the intracellular domain has a length of 10 to 700 amino acid residues. In embodiments, the intracellular domain has a length of 20 to 700 amino acid residues. In embodiments, the intracellular domain has a length of 30 to 700 amino acid residues. In embodiments, the intracellular domain has a length of 40 to 700 amino acid residues. In embodiments, the intracellular domain has a length of 50 to 700 amino acid residues. In embodiments, the intracellular domain has a length of 60 to 700 amino acid residues. In embodiments, the intracellular domain has a length of 70 to 700 amino acid residues. In embodiments, the intracellular domain has a length of 80 to 700 amino acid residues. In embodiments, the intracellular domain has a length of 90 to 700 amino acid residues. In embodiments, the intracellular domain has a length of 100 to 700 amino acid residues. In embodiments, the intracellular domain has a length of 150 to 700 amino acid residues. In embodiments, the intracellular domain has a length of 200 to 700 amino acid residues. In embodiments, the intracellular domain has a length of 250 to 700 amino acid residues. In embodiments, the intracellular domain has a length of 300 to 700 amino acid residues. In embodiments, the intracellular domain has a length of 350 to 700 amino acid residues. In embodiments, the intracellular domain has a length of 400 to 700 amino acid residues. In embodiments, the intracellular domain has a length of 450 to 700 amino acid residues. In embodiments, the intracellular domain has a length of 500 to 700 amino acid residues. In embodiments, the intracellular domain has a length of 550 to 700 amino acid residues. In embodiments, the intracellular domain has a length of 600 to 700 amino acid residues. In embodiments, the intracellular domain has a length of 650 to 700 amino acid residues.

In embodiments, the intracellular domain has a length of 10 to 650 amino acid residues. In embodiments, the intracellular domain has a length of 10 to 600 amino acid residues. In embodiments, the intracellular domain has a length of 10 to 550 amino acid residues. In embodiments, the intracellular domain has a length of 10 to 500 amino acid residues. In embodiments, the intracellular domain has a length of 10 to 450 amino acid residues. In embodiments, the intracellular domain has a length of 10 to 400 amino acid residues. In embodiments, the intracellular domain has a length of 10 to 350 amino acid residues. In embodiments, the intracellular domain has a length of 10 to 300 amino acid residues. In embodiments, the intracellular domain has a length of 10 to 250 amino acid residues. In embodiments, the intracellular domain has a length of 10 to 200 amino acid residues. In embodiments, the intracellular domain has a length of 10 to 150 amino acid residues. In embodiments, the intracellular domain has a length of 10 to 100 amino acid residues. In embodiments, the intracellular domain has a length of 10 to 90 amino acid residues. In embodiments, the intracellular domain has a length of 10 to 80 amino acid residues. In embodiments, the intracellular domain has a length of 10 to 70 amino acid residues. In embodiments, the intracellular domain has a length of 10 to 60 amino acid residues. In embodiments, the intracellular domain has a length of 10 to 50 amino acid residues. In embodiments, the intracellular domain has a length of 10 to 40 amino acid residues. In embodiments, the intracellular domain has a length of 10 to 30 amino acid residues. In embodiments, the intracellular domain has a length of 10 to 20 amino acid residues.

In another aspect is provided a transmembrane domain provided herein including embodiments thereof, wherein the transmembrane domain is covalently bound to the first intein through a covalent linker.

In embodiments, the linker includes a peptide linker, wherein the peptide linker is at least 3 amino acids in length. In embodiments, the peptide linker has a length of about 3 to about 20 amino acid residues. In embodiments, the peptide linker has a length of about 4 to about 20 amino acid residues. In embodiments, the peptide linker has a length of about 5 to about 20 amino acid residues. In embodiments, the peptide linker has a length of about 6 to about 20 amino acid residues. In embodiments, the peptide linker has a length of about 7 to about 20 amino acid residues. In embodiments, the peptide linker has a length of about 8 to about 20 amino acid residues. In embodiments, the peptide linker has a length of about 9 to about 20 amino acid residues. In embodiments, the peptide linker has a length of about 10 to about 20 amino acid residues. In embodiments, the peptide linker has a length of about 11 to about 20 amino acid residues. In embodiments, the peptide linker has a length of about 12 to about 20 amino acid residues. In embodiments, the peptide linker has a length of about 13 to about 20 amino acid residues. In embodiments, the peptide linker has a length of about 14 to about 20 amino acid residues. In embodiments, the peptide linker has a length of about 15 to about 20 amino acid residues. In embodiments, the peptide linker has a length of about 16 to about 20 amino acid residues. In embodiments, the peptide linker has a length of about 17 to about 20 amino acid residues. In embodiments, the peptide linker has a length of about 18 to about 20 amino acid residues. In embodiments, the peptide linker has a length of about 19 to about 20 amino acid residues.

In embodiments, the peptide linker has a length of about 3 to about 19 amino acid residues. In embodiments, the peptide linker has a length of about 3 to about 18 amino acid residues. In embodiments, the peptide linker has a length of about 3 to about 17 amino acid residues. In embodiments, the peptide linker has a length of about 3 to about 16 amino acid residues. In embodiments, the peptide linker has a length of about 3 to about 15 amino acid residues. In embodiments, the peptide linker has a length of about 3 to about 14 amino acid residues. In embodiments, the peptide linker has a length of about 3 to about 13 amino acid residues. In embodiments, the peptide linker has a length of about 3 to about 12 amino acid residues. In embodiments, the peptide linker has a length of about 3 to about 11 amino acid residues. In embodiments, the peptide linker has a length of about 3 to about 10 amino acid residues. In embodiments, the peptide linker has a length of about 3 to about 9 amino acid residues. In embodiments, the peptide linker has a length of about 3 to about 8 amino acid residues. In embodiments, the peptide linker has a length of about 3 to about 7 amino acid residues. In embodiments, the peptide linker has a length of about 3 to about 6 amino acid residues. In embodiments, the peptide linker has a length of about 3 to about 5 amino acid residues. In embodiments, the peptide linker has a length of about 3 to about 4 amino acid residues.

In embodiments, the peptide linker has a length of 3 to 20 amino acid residues. In embodiments, the peptide linker has a length of 4 to 20 amino acid residues. In embodiments, the peptide linker has a length of 5 to 20 amino acid residues. In embodiments, the peptide linker has a length of 6 to 20 amino acid residues. In embodiments, the peptide linker has a length of 7 to 20 amino acid residues. In embodiments, the peptide linker has a length of 8 to 20 amino acid residues. In embodiments, the peptide linker has a length of 9 to 20 amino acid residues. In embodiments, the peptide linker has a length of 10 to 20 amino acid residues. In embodiments, the peptide linker has a length of 11 to 20 amino acid residues. In embodiments, the peptide linker has a length of 12 to 20 amino acid residues. In embodiments, the peptide linker has a length of 13 to 20 amino acid residues. In embodiments, the peptide linker has a length of 14 to 20 amino acid residues. In embodiments, the peptide linker has a length of 15 to 20 amino acid residues. In embodiments, the peptide linker has a length of 16 to 20 amino acid residues. In embodiments, the peptide linker has a length of 17 to 20 amino acid residues. In embodiments, the peptide linker has a length of 18 to 20 amino acid residues. In embodiments, the peptide linker has a length of 19 to 20 amino acid residues.

In embodiments, the peptide linker has a length of 3 to 19 amino acid residues. In embodiments, the peptide linker has a length of 3 to 18 amino acid residues. In embodiments, the peptide linker has a length of 3 to 17 amino acid residues. In embodiments, the peptide linker has a length of 3 to 16 amino acid residues. In embodiments, the peptide linker has a length of 3 to 15 amino acid residues. In embodiments, the peptide linker has a length of 3 to 14 amino acid residues. In embodiments, the peptide linker has a length of 3 to 13 amino acid residues. In embodiments, the peptide linker has a length of 3 to 12 amino acid residues. In embodiments, the peptide linker has a length of 3 to 11 amino acid residues. In embodiments, the peptide linker has a length of 3 to 10 amino acid residues. In embodiments, the peptide linker has a length of 3 to 9 amino acid residues. In embodiments, the peptide linker has a length of 3 to 8 amino acid residues. In embodiments, the peptide linker has a length of 3 to 7 amino acid residues. In embodiments, the peptide linker has a length of 3 to 6 amino acid residues. In embodiments, the peptide linker has a length of 3 to 5 amino acid residues. In embodiments, the peptide linker has a length of 3 to 4 amino acid residues.

In embodiments, the peptide linker includes at least one glycine or one serine residue. In embodiments, the peptide linker includes at least one glycine residue. In embodiments the peptide linker includes at least one serine residue. In embodiments, the peptide linker includes one or more (e.g., 1, 2, 3, 4, 5, 6, 7) glycine amino acid residues. In embodiments, the peptide linker includes one or more (e.g., 1, 2, 3, 4, 5, 6, 7) serine amino acid residues.

In another aspect is provided a fusion protein including a transmembrane domain covalently bound to a biologically active protein domain through a first peptide linker, wherein the transmembrane domain is embedded within a phospholipid layer; and wherein the first peptide linker includes an intein scar amino acid sequence. In embodiments, the length of the intein scar is at least 2 amino acids. In embodiments, the length of the intein scar is at least 3 amino acids. In embodiments, the length of the intein scar is at least 4 amino acids. In embodiments, the length of the intein scar is at least 5 amino acids. In embodiments, the length of the intein scar is at least 6 amino acids. In embodiments, the length of the intein scar is at least 7 amino acids. In embodiments, the length of the intein scar is at least 8 amino acids. In embodiments, the length of the intein scar is at least 9 amino acids.

In embodiments, the intein scar amino acid sequence is the sequence of SEQ ID NO:7, SEQ ID NO:8, SEQ ID NO:9 or SEQ ID NO:10. In embodiments, the intein scar amino acid sequence is the sequence of SEQ ID NO:7. In embodiments, the intein scar amino acid sequence is the sequence of SEQ ID NO:8. In embodiments, the intein scar amino acid sequence is the sequence of SEQ ID NO:9. In embodiments, the intein scar amino acid sequence is the sequence of SEQ ID NO:10. In embodiments, the intein scar amino acid sequence is flanked by a peptide linker on either side of the scar. In further embodiments, the peptide linker is a polyglycine ([Gly]₁₋₁₀) or a polyglycine-polyserine ([GlySer]₁₋₁₀) sequence. In embodiments, the peptide linker is a polyglycine ([Gly]₁₋₁₀) sequence. In embodiments, the peptide linker is a polyglycine-polyserine ([GlySer]₁₋₁₀) sequence.

In embodiments, the transmembrane domain covalently is bound to a biologically active protein domain through the first peptide linker and a second linker. In embodiments, the second linker is N-terminal to the first peptide linker. In embodiments, the second linker is C-terminal to the first peptide linker. In embodiments, the second linker is C-terminal to the first peptide linker.

In embodiments, the second linker includes a second peptide linker, wherein the second peptide linker is at least 3 amino acids in length. In embodiments, the second linker has a length of about 3 to about 20 amino acid residues. In embodiments, the second linker has a length of about 4 to about 20 amino acid residues. In embodiments, the second linker has a length of about 5 to about 20 amino acid residues. In embodiments, the second linker has a length of about 6 to about 20 amino acid residues. In embodiments, the second linker has a length of about 7 to about 20 amino acid residues. In embodiments, the second linker has a length of about 8 to about 20 amino acid residues. In embodiments, the second linker has a length of about 9 to about 20 amino acid residues. In embodiments, the second linker has a length of about 10 to about 20 amino acid residues. In embodiments, the second linker has a length of about 11 to about 20 amino acid residues. In embodiments, the second linker has a length of about 12 to about 20 amino acid residues. In embodiments, the second linker has a length of about 13 to about 20 amino acid residues. In embodiments, the second linker has a length of about 14 to about 20 amino acid residues. In embodiments, the second linker has a length of about 15 to about 20 amino acid residues. In embodiments, the second linker has a length of about 16 to about 20 amino acid residues. In embodiments, the second linker has a length of about 17 to about 20 amino acid residues. In embodiments, the second linker has a length of about 18 to about 20 amino acid residues. In embodiments, the second linker has a length of about 19 to about 20 amino acid residues.

In embodiments, the second linker has a length of about 3 to about 19 amino acid residues. In embodiments, the second linker has a length of about 3 to about 18 amino acid residues. In embodiments, the second linker has a length of about 3 to about 17 amino acid residues. In embodiments, the second linker has a length of about 3 to about 16 amino acid residues. In embodiments, the second linker has a length of about 3 to about 15 amino acid residues. In embodiments, the second linker has a length of about 3 to about 14 amino acid residues. In embodiments, the second linker has a length of about 3 to about 13 amino acid residues. In embodiments, the second linker has a length of about 3 to about 12 amino acid residues. In embodiments, the second linker has a length of about 3 to about 11 amino acid residues. In embodiments, the second linker has a length of about 3 to about 10 amino acid residues. In embodiments, the second linker has a length of about 3 to about 9 amino acid residues. In embodiments, the second linker has a length of about 3 to about 8 amino acid residues. In embodiments, the second linker has a length of about 3 to about 7 amino acid residues. In embodiments, the second linker has a length of about 3 to about 6 amino acid residues. In embodiments, the second linker has a length of about 3 to about 5 amino acid residues. In embodiments, the second linker has a length of about 3 to about 4 amino acid residues.

In embodiments, the second linker has a length of 3 to 20 amino acid residues. In embodiments, the second linker has a length of 4 to 20 amino acid residues. In embodiments, the second linker has a length of 5 to 20 amino acid residues. In embodiments, the second linker has a length of 6 to 20 amino acid residues. In embodiments, the second linker has a length of 7 to 20 amino acid residues. In embodiments, the second linker has a length of 8 to 20 amino acid residues. In embodiments, the second linker has a length of 9 to 20 amino acid residues. In embodiments, the second linker has a length of 10 to 20 amino acid residues. In embodiments, the second linker has a length of 11 to 20 amino acid residues. In embodiments, the second linker has a length of 12 to 20 amino acid residues. In embodiments, the second linker has a length of 13 to 20 amino acid residues. In embodiments, the second linker has a length of 14 to 20 amino acid residues. In embodiments, the second linker has a length of 15 to 20 amino acid residues. In embodiments, the second linker has a length of 16 to 20 amino acid residues. In embodiments, the second linker has a length of 17 to 20 amino acid residues. In embodiments, the second linker has a length of 18 to 20 amino acid residues. In embodiments, the second linker has a length of 19 to 20 amino acid residues.

In embodiments, the second linker has a length of 3 to 19 amino acid residues. In embodiments, the second linker has a length of 3 to 18 amino acid residues. In embodiments, the second linker has a length of 3 to 17 amino acid residues. In embodiments, the second linker has a length of 3 to 16 amino acid residues. In embodiments, the second linker has a length of 3 to 15 amino acid residues. In embodiments, the second linker has a length of 3 to 14 amino acid residues. In embodiments, the second linker has a length of 3 to 13 amino acid residues. In embodiments, the second linker has a length of 3 to 12 amino acid residues. In embodiments, the second linker has a length of 3 to 11 amino acid residues. In embodiments, the second linker has a length of 3 to 10 amino acid residues. In embodiments, the second linker has a length of 3 to 9 amino acid residues. In embodiments, the second linker has a length of 3 to 8 amino acid residues. In embodiments, the second linker has a length of 3 to 7 amino acid residues. In embodiments, the second linker has a length of 3 to 6 amino acid residues. In embodiments, the second linker has a length of 3 to 5 amino acid residues. In embodiments, the second linker has a length of 3 to 4 amino acid residues.

In embodiments, the second peptide linker includes at least one glycine or one serine residue. In embodiments, the second peptide linker includes at least one glycine residue. In embodiments, the second peptide linker includes at least one serine residue. In embodiments, the second linker includes one or more (e.g., 1, 2, 3, 4, 5, 6, 7) glycine amino acid residues. In embodiments, the second linker includes one or more (e.g., 1, 2, 3, 4, 5, 6, 7) serine amino acid residues.

In another aspect is provided a kit composition including a transmembrane domain covalently bound to a first intein of a split intein pair, wherein the transmembrane domain is embedded within a phospholipid layer.

In an aspect, provided herein is a first polypeptide including a transmembrane domain covalently bound to a C-intein or N-intein. Also provided are vesicles that include such polypeptides.

In an aspect, provided herein is are compositions including a first polypeptide including a transmembrane domain covalently bound to a C-intein or N-intein and a second polypeptide covalently bound to a C-intein or N-intein, wherein the if the first polypeptide is bound to a C-intein then the second polypeptides is covalently bound to an N-intein, and wherein if the first polypeptide is bound to a N-intein then the second polypeptides is covalently bound to an C-intein. Also provided are vesicles that include such polypeptides

### III. Methods

In another aspect is provided a method of synthesis of a fusion protein, the method including: (a) contacting a transmembrane domain with a biologically active protein domain, wherein the transmembrane domain is covalently bound to a first intein of a split intein pair and the transmembrane domain is embedded within a phospholipid layer, wherein the biologically active protein domain is covalently bound to a second intein of the split intein pair, and (b) allowing the first intein to react with the second intein thereby forming the fusion protein. In embodiments, the fusion protein embedded in a phospholipid layer is made in the absence of detergent. Methods for reconstituting polypeptides in a phospholipid layer are well known in the art. See Shen H H, Lithgow T, Martin L. Reconstitution of membrane proteins into model membranes: seeking better ways to retain protein activities. Int J Mol Sci. 2013 Jan. 14; 14(1):1589-607.

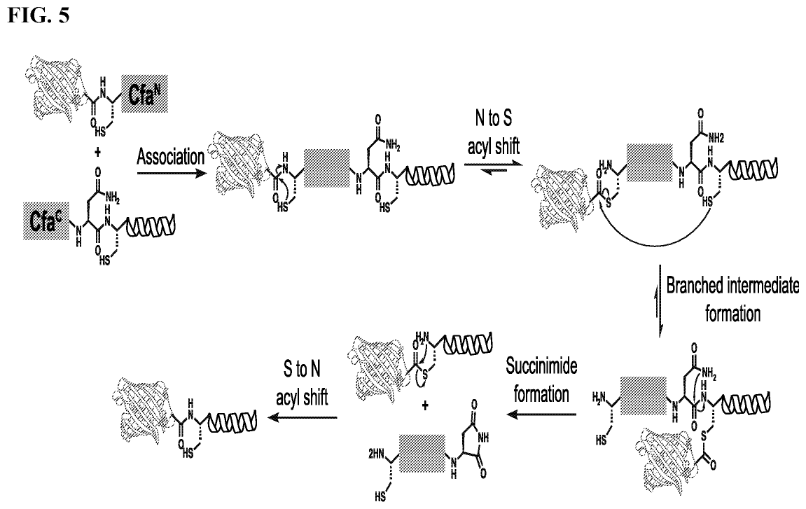

In embodiments, the reaction of the first and second intein is a transthioesterification reaction. See FIG. 5A for a schematic showing the reaction. In embodiments, the phospholipid layer is a lipid vesicle, a nanodisc, a lipid nanoparticle, or a polymersome. In embodiments, the phospholipid layer is a lipid vesicle. In embodiments, the phospholipid layer is a nanodisc. In embodiments, the phospholipid layer is a lipid nanoparticle. In embodiments, the phospholipid layer is a polymersome. In embodiments, the phospholipid layer forms part of a lipid vesicle, a nanodisc, a lipid nanoparticle, or a polymersome. In embodiments, the phospholipid layer forms part of a lipid vesicle. In embodiments, the phospholipid layer forms part of a nanodisc. In embodiments, the phospholipid layer forms part of a lipid nanoparticle. In embodiments, the phospholipid layer forms part of a polymersome.

In embodiments, the first intein is a C-intein or an N-intein. In embodiments, the second intein is a C-intein or an N-intein. Split inteins are well known in the art. See Aranko A S, Wlodawer A, Iwaï H. Nature's recipe for splitting inteins. Protein Eng Des Sel. 2014 August; 27(8):263-71. In embodiments, the split intein is a C-intein or N-intein from Cfa, PhoRadA, RmaDnaB^{Δ286}, SspDnaB^{Δ275}, SspDnaX, TvoVMA, NpuDnaE, NpuDnaB^{Δ283}, SspGyrB, TerThyX, AceL-TerL, PchPRP8, PfuRIR1-1, Psp-GDBPol-1, PfuRIR1-2, SceVMA^{Δ206}, RmaDnaB^{Δ27}1, MtuRecA^{Δ285}, SspDnaB^{Δ274}, gp41-8, SceVMAA²²⁷, IMPDH-1, NrdJ-1, MtuRecA^{Δ297}, gp41-1, AovDnaE, AspDnaE, AvaDnaE, Cra(C5505)DnaE, Csp(CCY0110)DnaE, CwaDnaE, Maer(NIES843)DnaE, Mcht(PCC7420)DnaE, MtuRecA^{Δ300}, NspDnaE, OliDnaE, Sel(PC7942)DnaE, Ssp(PCC7002)DnaE, TerDnaE-3, TelDnaE, TvuDnaE, NeqPol, or TerThyX^{Δ132}. In embodiments, the transmembrane domain is covalently bound to the first intein through a first covalent linker. In embodiments, the first covalent linker includes a first peptide linker, wherein the first peptide linker is at least 3 amino acids in length. In embodiments, the first peptide linker includes at least one glycine or one serine residue. In embodiments, the first peptide linker includes at least one glycine residue. In embodiments, the first peptide linker includes at least one serine residue. In embodiments, the biologically active protein domain is covalently bound to the second intein through a second covalent linker. In embodiments, the second covalent linker includes a second peptide linker, wherein the second peptide linker is at least 3 amino acids in length. In embodiments, the second peptide linker includes at least one glycine or one serine residue. In embodiments, the second peptide linker includes at least one glycine residue. In embodiments, the second peptide linker includes at least one serine residue. In embodiments, the transmembrane domain is a synthetic WALP or a transmembrane domain of a signaling, receptor, channel, transport, or G-protein coupled receptor (GPCR) membrane protein. In embodiments, the biologically active polypeptide domain is fragment of a protein that facilitates binding, signaling, enzymatic function, transport, synthesis, stability, or other functional biological function.

In an aspect, provided herein are methods of synthesis of a transmembrane polypeptide by contacting a first polypeptide including a transmembrane domain covalently bound to a C-intein with a second polypeptide covalently bound to an N-intein or contacting the first polypeptide covalently bound to a N-intein with the second polypeptide covalently bound to an C-intein. Also provided are methods further including reconstituting the first polypeptide in a vesicle.

### EXAMPLES

### Example 1: Method for Transmembrane Protein Semisynthesis and Reconstitution in Lipid Membranes

Presented herein are novel methods for the semisynthesis of transmembrane (TM) proteins in lipid membranes. Semisynthesis of single pass transmembrane proteins on liposomes and giant unilamellar vesicles (GUVs) were obtained by using split intein ligation. Split inteins are natural or engineered protein trans-splicing domains. By leveraging the biorthogonality and chemoselectivity of split inteins, overexpressed soluble domains are ligated to synthetic transmembrane peptides to build semisynthetic membrane proteins directly on phospholipid vesicles. This one-pot method bypasses the painstaking expression of recombinantly expressed integral membrane proteins and the multistep process of detergent-based protein reconstitution, making it easier to study these important biomolecules in an isolated system.

Cellular lipid membranes are embedded with transmembrane proteins crucial to cell function. Elucidating membrane proteins' diverse structures and biophysical mechanisms is increasingly necessary due to their growing prevalence as a therapeutic target and sheer ubiquity in cells. Most biophysical characterization strategies of transmembrane proteins rely on the tedious overexpression and isolation of recombinant proteins and their reconstitution in model phospholipid bilayers. Unfortunately, membrane protein reconstitution depends on the use of denaturing and unnatural detergents that may interfere with protein structure and function. A detergent-free method is provided to reconstitute transmembrane proteins in model phospholipid vesicles and GUVs. Additionally, transmembrane proteins are difficult to express in cells due to the extreme insolubility of their transmembrane domain. By incorporating a synthetic transmembrane peptide into liposomes and simply expressing soluble portions of transmembrane proteins in cells, semisynthetic ligation strategy can be used to construct functional transmembrane proteins and reconstitute them into liposomes for biophysical and biochemical studies.

Inteins can be found contiguously or non-contiguously within some proteins. Non-contiguous inteins are called “split inteins”. Inteins can be thought of as a type of protein intron which splices itself out of proteins. When non-contiguous inteins find and bind to each other, they are then able to excise themselves resulting in the ligation of their respective exteins. Split intein pairs (C-intein and N-intein) can be attached to proteins of interest in synthetic and cellular systems to ligate protein sequences together. Here, a synthetic transmembrane (TM) peptide, of a natural or unnatural sequence, fused to a C-intein construct is synthesized via solid phase peptide synthesis. A soluble protein or soluble domain of a transmembrane protein is expressed in cells as a recombinant protein-N-intein fusion. The TM peptide is incorporated into liposomes by making a phospholipid (1,2-dioleoyl-sn-glycero-3-phosphatidylcholine (DOPC))+TM peptide film and hydrating it in water or buffer. Multilamellar vesicles with incorporated TM peptide are made via simple hydration while GUVs with incorporated TM peptide are made via electroformation. The soluble protein-intein fusion is added to the peptide-loaded vesicles and the ligation reaction proceeds on the phospholipid membrane: split intein association results in an N to S acyl shift. A transthioesterification results in the formation of the branched intermediate. Succinimide formation releases both inteins and a final S to N acyl shift results in the ligated extein product (in this invention, a transmembrane peptide fused with soluble proteins or protein domains) with a native peptide bond. Subsequent SDS-PAGE, microscopy, and mass spectra of the product can be used to verify that the reaction has taken place.

GFP was ligated to a synthetic transmembrane peptide using this strategy in murine leukemia viruses (MLVs) and GUVs. The successful synthesis product was verified by mass spec, SDS-PAGE, and colocalization via confocal fluorescence microscopy. Additionally, a biologically relevant protein, programmed cell death protein 1 (PD-1), was ligated to synthetic peptides using this strategy. Functional studies of semisynthesized PD-1 in GUVs are also performed.

### Example 2: Semisynthesis of Functional Transmembrane Proteins in GUVs

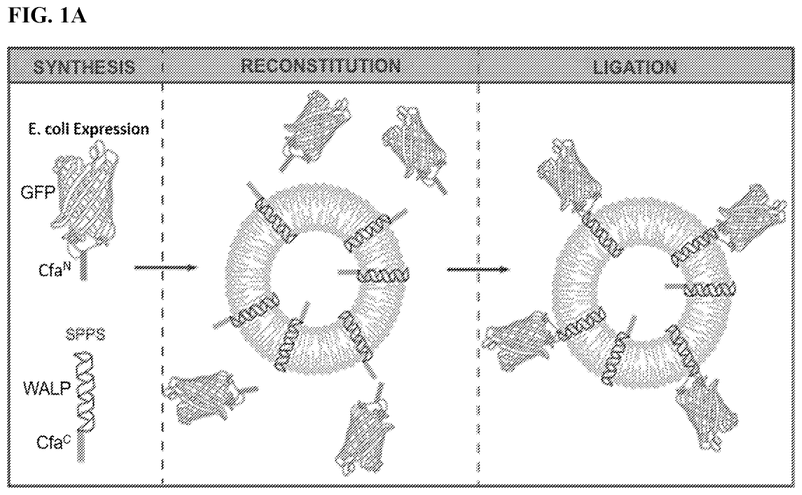

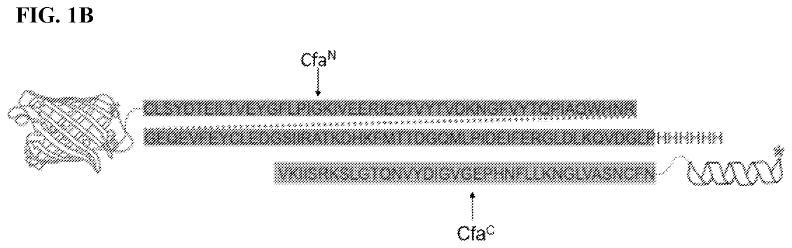

Identification of a suitable split intein-mediated ligation system compatible with semisynthetic reactions was done. The engineered Cfa_{GEP} split intein system, derived from the ultrafast Cfa_{WT}, was chosen for its improved extein tolerance which enables versatility in protein semisynthesis.ref Cfa_{GEP} is reportedly robust for semisynthesis, contains a small C intein (38 amino acids) ideal for peptide synthesis, and results in minimal amino acid scaring between exteins.ref. Using Cfa_{GEP}, further designing of a proof-of-concept semisynthetic pair, a protein extein fused to the N intein (protein-Cfa^{N}) and peptide extein fused to the C intein (Cfa^{C}-peptide) were done, capable of ligating in phospholipid membranes (FIG. 1A). Green fluorescent protein (GFP) was chosen as the protein extein as an easily recombinantly expressed protein with fluorescent properties useful for downstream imaging experiments. GFP fused to Cfa^{N} with a C-terminal polyhistidine tag (GFP-Cfa^{N}-His₆) was expressed in E. coli and purified by Ni-NTA column (FIG. 1B). A well-characterized, single-pass transmembrane (TM) peptide known as a WALP was chosen as a model synthetic transmembrane peptide extein. WALPs classically contain leucine and alanine (LA) repeats flanked by two tryptophans (WW) on each terminus. A Cfa^{C}-WALP peptide was produced via solid phase peptide synthesis (SPPS) on a peptide synthesizer (CEM Liberty Blue; FIG. 1B). A fluorescent derivative of Cfa^{C}-WALP containing a lysine side chain conjugated to carboxyfluorescein (Cfa^{C}-WALP-CF) was also synthesized. After purification of the TM peptides on a reverse phase C-18 column, liquid chromatography electrospray ionization time of flight mass spectrometry (LC-ESI-TOFMS) confirmed their purity and mass.

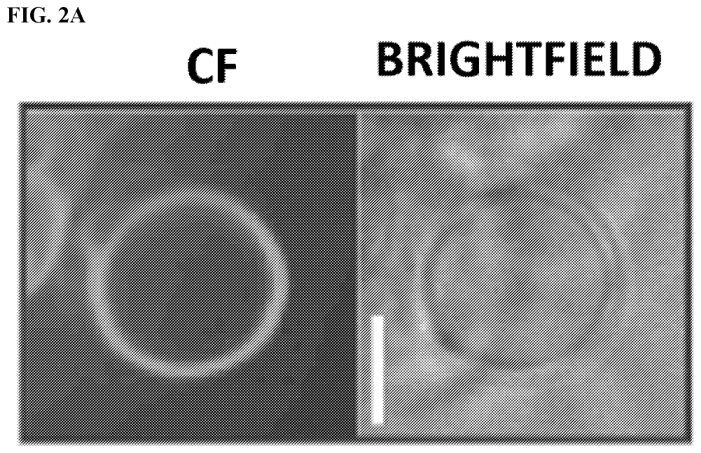

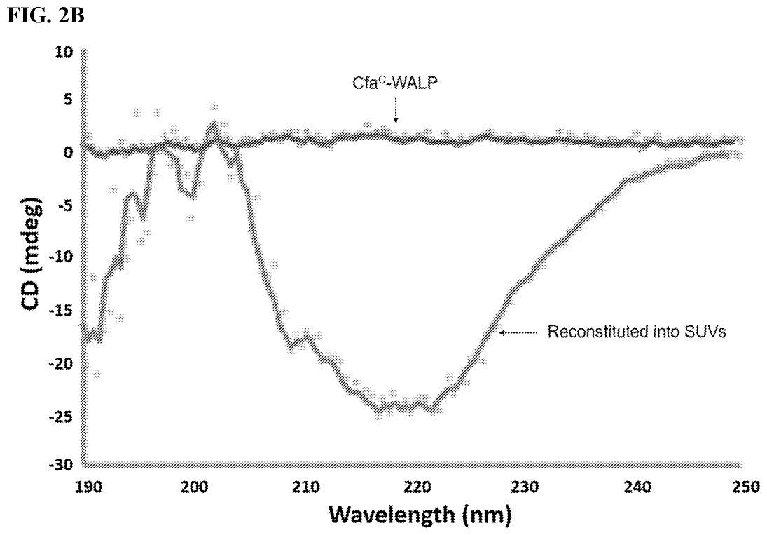

Reconstitution of the TM peptide into phospholipid bilayers was verified. A lipid and Cfa^{C}-WALP-CF (50:1) film was made under a stream of nitrogen gas and subsequently hydrated with water or buffer to reconstitute the TM peptide into phospholipid membranes. Confocal fluorescence microscopy confirms the localization of Cfa^{C}-WALP-CF to hydrated DOPC vesicles (FIG. 2A). Cryogenic transmission electron microscopy (cryo-TEM) showed no disruption of the lipid membranes by peptide incorporation and no visible accumulation of peptide at vesicle surfaces indicating its reconstitution into DOPC membranes. Circular dichroism (CD) spectra of Cfa^{C}-WALP and Cfa^{C}-WALP-CF inserted in DOPC corroborates previously published WALP CD spectra showing that the peptide is in an unfolded, disordered state alone, but folds into a secondary alpha helix structure once reconstituted into DOPC unilamellar vesicles (FIG. 2B). These results show that the synthetic Cfa^{C}-WALPs reconstitute into DOPC bilayers as alpha helical TM peptides.

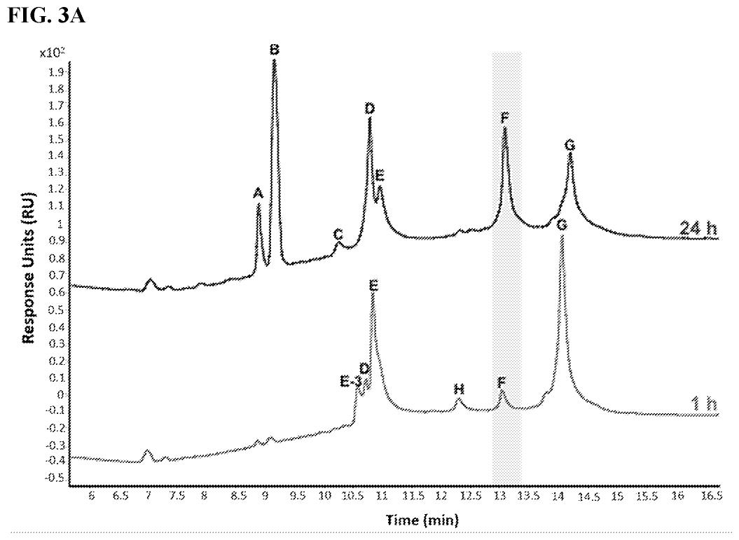

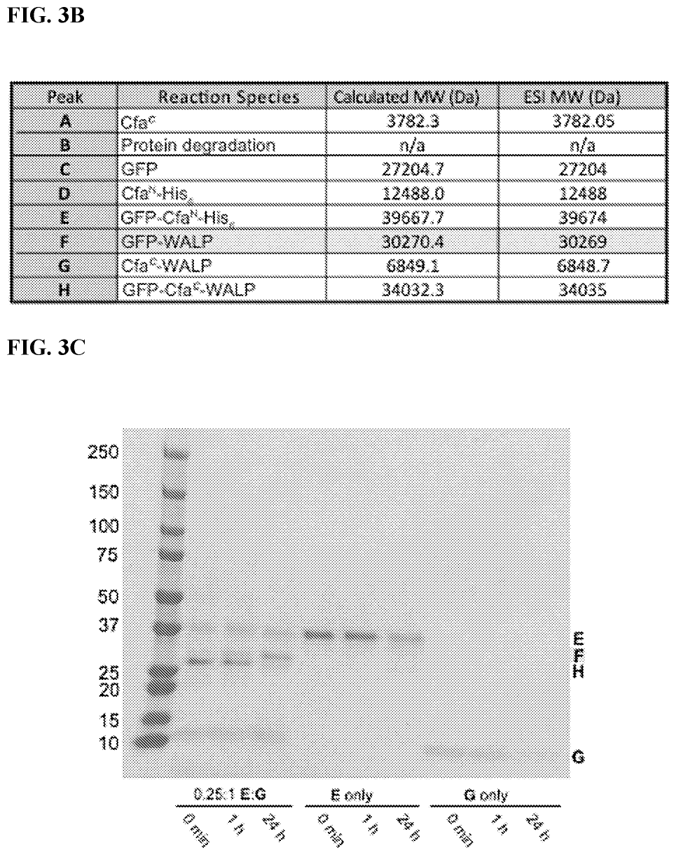

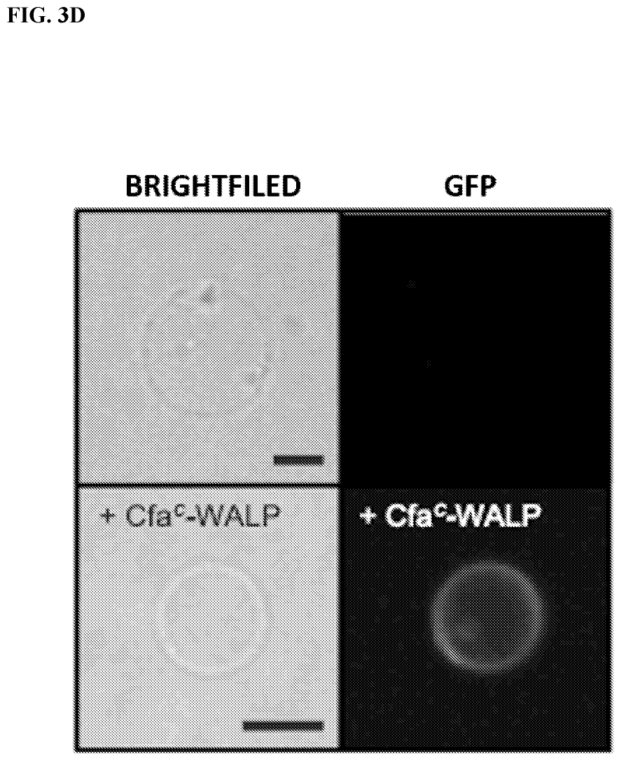

Split intein-mediated GFP-WALP ligation in phospholipid membranes was demonstrated. Mimicking previously established conditions for Cfa_{GEP} splicing, soluble GFP-Cfa^{N}-His6 was reacted with liposome-reconstituted Cfa^{C}-WALP (2:1) in splicing buffer (150 mM sodium phosphates, 100 mM NaCl, 5 mM EDTA, 1 mM TCEP pH 7). The predicted 30.2 kDa product is GFP-WALP with an eight amino acid ligation scar (GGCFNGGG) between the GFP and WALP. LC-ESI-TOFMS analysis confirmed the presence and expected mass of GFP-WALP product, F, in the reaction mixture after 1 and 24 h (FIGS. 3A-3B). SDS-PAGE corroborated these results, where even at 0 min, there is a rapid conversion of all reacting GFP-Cfa^{N}-His₆, E, into an intermediate, H (FIG. 3C). LC-ESI-TOFMS reveals that H is a covalently-linked, branched intermediate of the intein ligation (GFP-Cfa^{C}-WALP). The gel band intensities of H and F show the conversion of H to F over time. Taken together, the LC-ESI-TOFMS and SDS-PAGE data indicate that the semisynthetic split intein-mediated ligation of protein and TM peptide is taking place in liposomes.

Large unilamellar bilayers are necessary for liposomal models of cellular processes, so we sought to bring this ligation system into giant unilamellar vesicles (GUVs). We turned to electroformation as a common method of GUV formation that does not use protein structure- and function-altering detergents. Unfortunately, common electroformation methods using indium tin oxide-coated glass slides are incompatible with high salt buffer such as the splice buffer essential to carry out the intein-mediated ligation reaction in GUVs. It was necessary to turn to alternative simultaneous electroformation and reconstitution methods compatible with the splice buffer. There has been success reconstituting expressed membrane proteins in high salt buffer into GUVs using platinum (Pt) wires and sequential changes in voltage sine wave parameters. With this electroformation procedure, we successfully reconstituted Cfa^{C}-WALP into GUVs in splice buffer. The ligation was tracked via confocal fluorescence microscopy where GFP-Cfa^{N}-His₆ reacted with TM peptide in GUVs (FIG. 3D). Controls confirmed that the GFP construct does not nonspecifically bind to DOPC membranes.

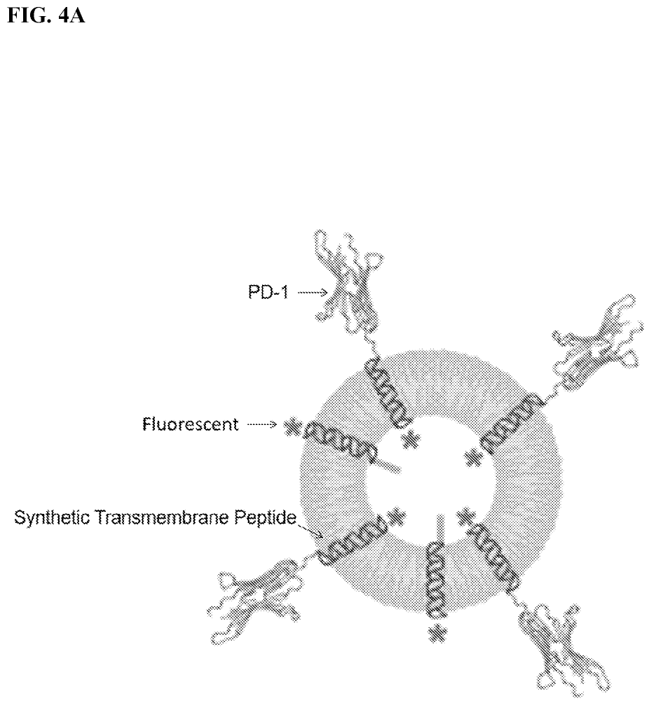

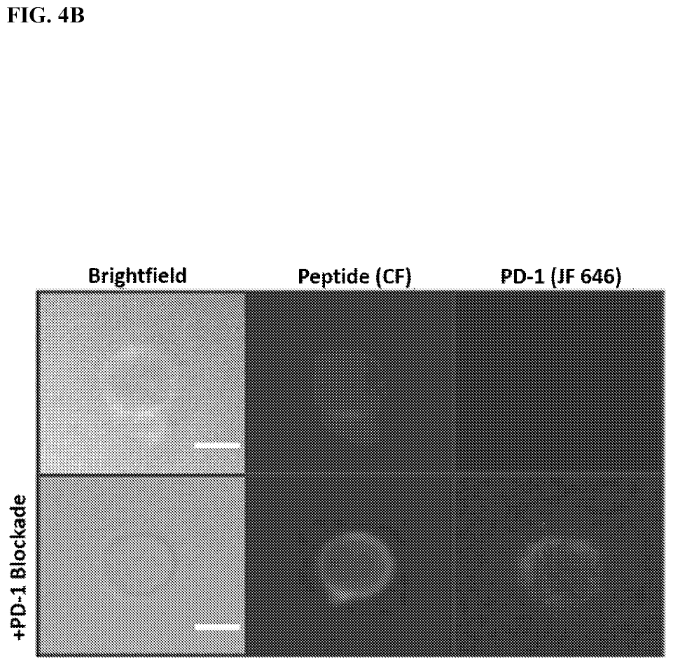

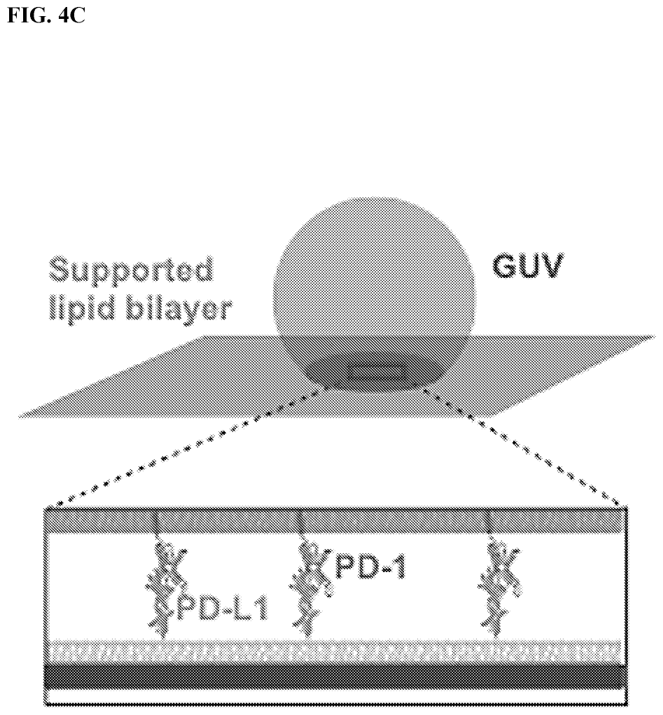

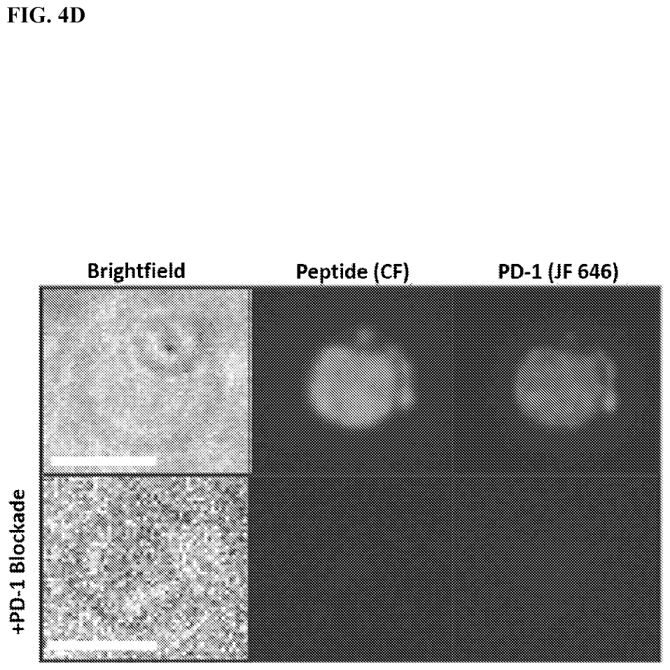

Programmed cell death protein 1 (PD-1) is single pass TM checkpoint protein that regulates T and B cell function by binding to PD-L1 on cells. As a central immune checkpoint target for cancer therapeutics, the PD-1/PD-L1 signaling pathway is an urgent focus for translation research. As with most TM proteins, full length PD-1 is challenging to express and reconstitute into model membranes for study. Fully glycosylated extracellular domain of PD-1 fused to Cfa^{N} in mammalian cells was expressed and purified. The recombinant protein was labeled with Janelia Fluor 646 (JF) for downstream fluorescence microscopy experiments and purified using standard procedures. Although the unpredictable glycosylations of PD-1 result in smeared starting material and product bands which are challenging to interpret by SDS-PAGE, the JF-PD-1-Cfa^{N} protein construct was reacted with Cfa^{C}-WALP-CF in GUVs and the successful ligation was easily monitored via fluorescence microscopy (FIG. 4A). These results indicate that the glycosylated extracellular domain of PD-1 is able to ligate to a TM peptide embedded in lipid membranes and does not nonspecifically bind to DOPC membranes.

To show that the extracellular domain of PD-1 remains functional after split intein-mediated semisynthesis, Total Internal Reflection Fluorescence (TIRF) microscopy was used to visualize microcluster formation upon PD-1 binding to PD-L1. Following standard procedures, polyhistidine-tagged PD-L1 were conjugated to supported lipid bilayers (SLBs) doped with 5% NTA-DGS lipids. GUVs containing semisynthetic Cfa^{C}-WALP-CF were electroformed in splice buffer and reacted with JF-PD-1-Cfa^{N}. These PD-1-WALP reconstituted GUVs were added to PD-L1 conjugated SLBs. The contact area between GUV and SLB was visualized by TIRF fluorescence microscopy for microcluster formation of PD-1 (FIG. 4B). As a control, to one well, a PD-1 antibody blockade was added to inhibit the binding of PD-1 to PD-L1. In the absence of blockade, the PD-1 and TM peptide fluorescent signals are enriched at the SLB-GUV interface. In the presence of PD-1 blockade, the sunken GUV is seen unable to bind but remains in close proximity to the SLB, indicated by the brightfield image of the bottom of the GUV and the minor PD-1 fluorescent signal. These results indicate that semisynthetic PD-1 retains its function after ligation to TM peptides within GUV membranes.

### Example 3: Materials, General Methods, and Instrument Details

Commercially available 1,2-dioleoyl-sn-glycero-3-phosphocholine (DOPC) was obtained from Avanti® Polar Lipids. Fmoc-Ala-OH, Fmoc-Arg(Pbf)-OH, Fmoc-Asn(trt)-OH, Fmoc-Asp(OtBu)-OH, Fmoc-Cys(trt)-OH, Fmoc-Gln(trt)-OH, Fmoc-Glu(OtBu)-OH, Fmoc-Gly-OH, Fmoc-His(trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, and Fmoc-Val-OH were purchased from ChemImpex. Fmoc-Lys(5/6-FAM)-OH was purchased from AnaSpec. N,N-dimethylformamide (DMF), acetonitrile (ACN), N,N-diisopropylethylamine (DIEA), trifluoroacetic acid (TFA), triisopropylsilane (TIS), 2-2′-(ethylenedioxy)diethanethiol (DODT), N,N′-diisopropylcarbodiimide (DIC), tris(2-carboxyethyl)phosphine hydrochloride (TCEP), 4-methylpiperidine, chloroform, anhydrous dichloromethane (DCM), anhydrous diethylether, and anhydrous methanol (MeOH) were obtained from Sigma-Aldrich. Oxyma was purchased from CEM. All reagents obtained from commercial suppliers were used without further purification unless otherwise noted. Spinning-disk confocal microscopy images were acquired on a Yokagawa spinning disk system (Yokagawa, Japan) built around an Axio Observer Z1 motorized inverted microscope (Carl Zeiss Microscopy GmbH, Germany) with a 63×, 1.40 NA oil immersion objective or 20×0.8 NA objective to an ORCA-Flash 4.0 V2 Digital CMOS camera (Hamamatsu, Japan) using ZEN Blue imaging software (Carl Zeiss Microscopy GmbH, Germany). The fluorophores were excited with diode lasers (405 nm-20 mW, 488 nm-30 mW, 561 nm-20 mW, and 638 nm-75 mW). A condenser/objective with a phase stop of Ph3 was used to obtain the phase-contrast images with a 20× objective on an Olympus BX51 upright fluorescent microscope. The fluorophores were excited with 20 mW DPSS lasers (GFP, JF 646). For cryo-TEM analysis, vesicles were imaged on a Titan Krios G3 transmission electron microscope (ThermoFisher) operated at 300 kV with an energy filter (Gatan), and volta phase plates. Images were recorded on a K2 Summit direct electron detector (Gatan). HPLC purification was carried out on Zorbax SB-C18 semipreparative column with Phase A/Phase B gradients [Phase A: H2O with 0.1% v/v TFA; Phase B: ACN with 0.1% v/v TFA] and monitored via diode array detector at 210 nm. Electrospray Ionization-Mass spectra (ESI-MS) were obtained on an Agilent 6230 Accurate-Mass Time of Flight (TOF) mass spectrometer from the UCSD Mass Spec Facility. Circular dichroism (CD) experiments were done on an AVIV CD Spectrometer Model 215. Scans were taken of each sample from 190-250 nm with a 1.0 nm Bandwidth in triplicate at room temperature.

### Experimental Procedures

Cloning GFP-Cfa^{N}-His₆: pTXB1, containing the Cfa^{N}_His₆ split intein gene, was kindly provided to us by Professor Dr. Galia Debolouchina's lab at the University of California, San Diego. A pET-11a cloning vector containing the sequence of the sfGFP construct was provided by Dr. Henrike Niederholdtmeyer in our lab. PCR and isothermal assembly (NEBuilder, New England Biolabs) was used per the vendor's instructions to insert the split intein gene into pET-11a, yielding a GFP-Cfa^{N}-His₆ fusion construct which was transformed into DH5α E. coli competent cells. A double glycine linker was placed between GFP-Cfa^{N} for improved splicing efficiency. After overnight culture at 37° C. and 200 rpm, plasmid minipreps were performed (Qiagen), and construct sequence was verified by Sanger Sequencing (Eton Biosciences).

Expression and purification of GFP-Cfa^{N}-His₆: Plasmids confirmed to have the correct fusion construct sequence were transformed into BL21 (DE3) E. coli competent cells (New England Biolabs) per vendor instructions. These cells were then grown overnight at 37° C. in Luria-Bertani (LB) broth containing 0.1 mg/mL carbenicillin, a more stable substitute antibiotic of ampicillin. 1 mL of the overnight culture was used to inoculate 100 mL of autoclaved LB medium containing 0.1 mg/mL carbenicillin. The culture was grown at 37° C. with shaking at 200 rpm until the OD600 of the culture reached 0.6. Overexpression of GFP-Cfa^{N}-His6 was induced with 0.5 mM isopropyl 1-thio-D-galactopyranoside (IPTG). The cells were then grown for 4 h at 37° C. with shaking at 200 rpm and subsequently harvested via centrifugation at 4000 ref for 20 min at 4° C. The visibly bright green (indicating the presence of full-length GFP) pellet was stored at −80° C. until further use. Buffers were prepared as followed: buffer A (50 mM phosphates, 300 mM NaCl, 5 mM imidazole, pH 7.5), wash buffer I (50 mM phosphates, 300 mM NaCl, 20 mM imidazole, pH 7.5), wash buffer II (50 mM phosphates, 200 mM NaCl, 50 mM imidazole, pH 7.5), elution buffer (50 mM phosphates, 300 mM NaCl, 250 mM imidazole, pH 7.5). Cell pellets were thawed and resuspended in lysis buffer (5 mL buffer A, 1 mM PMSF in ethanol) on ice. The resuspended cells were lysed on ice by ultrasonication (35% amplitude for 3 minutes 50% duty cycle with 40 second period at power level 6). The visibly bright green supernatant was incubated in a gravity column containing Ni2+− nitrilotriacitate (NTA) resin pre-equilibrated with 10 mM imidazole on a shaker for 1 h at 4° C. A small sample of flow through was kept for SDS-PAGE while the rest was discarded. The resin was washed four times on ice with 2 column volumes (CV; 600 μL) of wash buffer I and two times with 1 CV of wash buffer II by centrifuging the column for 2 seconds at 600 rcf into prepared tubes for collection of the supernatant. The column was washed six times on ice with 200 μL of elution buffer by gravity, each time collecting the visibly bright green eluent fraction in separate Eppendorf tubes. The fractions were analyzed by SDS-PAGE to check for considerable impurities. Fractions were pooled and aliquoted into high and low concentration samples to final concentrations of 19 μM and 373.5 μM. LC-ESI-TOFMS corroborated the purity and verified the correct mass of the protein construct.

### Expression, Purification, and Fluorescent Labeling of JF-PD-1-Cfa^{N}

SDS-PAGE: All SDS-PAGE experiments were ran for 35 minutes at 200 V on 15 well 4-20% MiniPROTEAN TGX Precast Protein Gels. Sample was added to loading dye (1:1) at specified time points, then placed on a 95° C. heat block for 5 minutes, placed on ice, quickly spun down via tabletop centrifuge, and loaded onto the gel. Gels were stained with Instant Blue Coomassie Stain (Abcam) for 1-24 h and destained with water. Gels were imaged on a tabletop scanner.

SDS-PAGE: All SDS-PAGE experiments were ran for 35 minutes at 200 V on 15 well 4-20% MiniPROTEAN TGX Precast Protein Gels. Sample was added to loading dye (1:1) at specified time points, then placed on a 95° C. heat block for 5 minutes, placed on ice, quickly spun down via tabletop centrifuge, and loaded onto the gel. Gels were stained with Instant Blue Coomassie Stain (Abcam) for 1-24 h and destained with water. Gels were imaged on a tabletop scanner.

Solid phase peptide synthesis (SPPS) of TM peptide: Cfa^{C}-WALP and Cfa^{C}-WALP-CF were synthesized in house via SPPS on a Liberty Blue peptide synthesizer (CEM) at a 0.1 mmol synthesis scale. A triple glycine linker was placed between Cfa^{C}-WALP in both constructs for improved splicing efficiency. Prior to synthesis, amino acids (0.2 M in DMF), coupling agent (20% v/v solution of 4-methylpiperidine in DMF), wash solvent (DMF), activator (0.5 M DIC in DMF), and base activator (0.5 M Oxyma and 0.1 eq DIEA in DMF), and pre-loaded resin were prepared. After swelling in anhydrous DCM for 10 minutes, 0.2 g Trityl-OH resin (ChemMatrix) was activated in 3 M acetyl chloride in DCM for 3 min at room temperature with shaking. The resin (0.5 mmol/g loading capacity) was then washed with anhydrous DCM (3×3 mL) and loaded with an amino acid solution containing 4 eq Fmoc-Ala-OH and 4 eq DIPEA in 2 mL DCM was added. The resin was shaken at room temperature overnight. It was drained and a capping solution of DCM/MeOH/DIEA (17:2:1) was added for 5 min with shaking at room temperature. The resin was then washed (3×2 CV of DCM, 2×2 CV of DMF, and 3×2 CV of DCM) and put on a desiccator to dry until placed in a 30 mL Liberty Blue reaction vessel for synthesis. Resin loading for both peptides were calculated to be ˜0.4 mmol/g resin using standard UV absorption method upon Fmoc cleaving of small aliquots of loaded resin. (ref) Subsequent protected amino acid couplings were done on the Liberty Blue peptide synthesizer using standard microwave-assisted deprotection and coupling settings. The 20 N-terminal amino acids were double-coupled to ensure coupling to the long-sequence, hydrophobic peptide. After synthesis, the peptide-conjugated resin was removed from the coupling vessel, washed with 3×2 CV of DCM, covered with foil, and desiccated overnight. To prepare the fluorescent Cfa^{C}-WALP-CF peptide (rather than the nonfluorescent Cfa^{C}-WALP), the C-terminal standard protected lysine was replaced with Fmoc-Lys(5/6-FAM)-OH (AnaSpec) during synthesis.

Deprotection and purification of TM peptide: Peptide-conjugated resin was shaken for 2 h at room temperature in a 6 mL TFA/TIS/H2O/DODT deprotection solution (37:1:1:1). The filtrate was collected in 15 mL Falcon tubes. Ice cold diethylether was added to each Falcon tube to precipitate the crude peptide product. The tubes were centrifuged at 7500 rcf for 5 min and the supernatant was discarded. The pellet was resuspended in ice cold anhydrous diethylether, centrifuged, and the supernatant was thrown out two additional times. The pellet was desiccated for 30 min and then dissolved in 0.5 mL H2O/methanol (1:1) and transferred to a weighed glass vial. To the dissolved crude peptide, 1 mL of H2O was added, and the peptide solution was frozen at −80° C. for lyophilization overnight. The lyophilized peptide powder was resuspended in H2O/MeOH (1:1) for HPLC purification (Zorbax SB-C18 semipreparative column, 5% v/v H2O+0.1% v/v TFA in ACN+0.1% v/v TFA; 10-11 min). The purified fraction was concentrated, lyophilized, and obtained as a white or yellow powder for Cfa^{C}-WALP and Cfa^{C}-WALP-CF, respectively. For experiments, 200 μM purified TM peptide stock solutions are freshly prepared in chloroform, vials sealed with parafilm, and stored at −20° C. for up to two weeks.

Reconstitution of TM peptide in MLVs: To reconstitute TM peptides into MLVs, a standard hydration method for vesicle formation was used. DOPC (25 μL, 10 mM) and TM peptide (25 μL, 200 μM) were mixed (50:1 lipid/peptide) and dried into a lipid and TM peptide film by N2 gas stream in a glass scintillation vial. The vial was desiccated for 30 min. Water or splice buffer (250 μL) was added to the vial which was then rotated at room temperature for 1 hour and vortexed. Confocal microscopy verified the formation of vesicles and the localization of TM fluorescent peptide to the vesicle membranes.

Reconstitution of TM peptide in GUVs: Previously published methods were adapted for GUV formation in high salt buffers to reconstitute TM peptides into GUVs. First, hydrated vesicle samples of a 50:1 lipid/peptide ratio were prepared in water as described above. These were sonicated in a bath sonicator for 1 h to form SUVs. In a Pt wire electroformation device compatible with confocal microscopy, seven drops of SUVs were placed on each Pt wire. The device was placed at 40° C. until the droplets were dried (˜5 min). Seven drops of SUVs were placed on each Pt wire and dried again at 40° C. for −5 min. The device was placed in a 40° C. chamber either on or off of the confocal microscope stage for monitoring the electroformation. Splice buffer (800 μL) was added right before a 60 MHz DDS signal generator (Koolerton) applied specific voltage and frequency to the electroformation device over a 3.5 h time period. After electroformation, GUVs were gently lifted off by gently tapping the device and pipetting splice buffer from inside the device over the wires ten (10) times. Before pipetting, the 1 cm of the pipette tip was snipped off with scissors to prevent GUV collapse due to shear forces within the smaller opening of the tip. The detached GUVs were visualized by spinning-disk confocal or compound microscopy.

Cryo-TEM of DOPC membranes with reconstituted TM peptide: MLVs with and without TM peptide reconstitution were prepared as described above. MLVs were sonicated in a bath sonicator for 1 h. Immediately before grid vitrification, the sample was pipetted onto plasma-cleaned 200-mesh Quantifoil R 2/2 copper grids (Quantifoil). Using a Vitrobot EM grid plunger (FEI), excess buffer was blotted at room temperature and 95% humidity, and the grids were plunge-frozen in liquid ethane maintained at −180° C. The grids were stored in liquid nitrogen until use. After clipping under cryogenic conditions and placing them in the autoloader cassette, the grids were loaded onto a Titan Krios for data collection using EPU (ThermoFisher) and DigitalMicrograph (Gatan) softwares.

Circular Dichroism: We adapted previous methods for analyzing the folding of WALPS reconstituted into lipid membranes via CD. Briefly, TM peptide reconstituted in SUVs were prepared reconstituting TM peptide in MLVs in water as described above (30:1 lipid/peptide ratio) and ultrasonicating the MLV sample for 3 minutes on ice (40% amplitude, power level 6). The SUV samples were spun down at 15000 rpm at 24° C. for 5 minutes and the supernatant was used for CD measurement. TM peptide samples without DOPC present were prepared by making a peptide film and hydrating the sample in water (final concentration 20 M).

Expression and purification of JF-PD-1-Cfa^{N}: The ectodomain of human PD-1 (aa 24-170) with an N-terminal signal peptide of HIV envelop glycoprotein gp120 followed by a SNAP-tag, and with a C-terminal Cfa^{N} followed by a TwinStrep-tag (PD-1-Cfa^{N}) was cloned into a pPPI4 plasmid and expressed in HEK293F cells. The secreted proteins were purified through StrepTrap HP column (GE Healthcare, 28907547) and labeled with JaneliaFluor646-conjugated SNAP ligand (JF, Janelia research). The labeled monomeric proteins were further purified using a Superdex 200 increase 10/300 GL column (GE Healthcare, 28990944) in HEPES buffered saline (50 mM HEPES, pH 7.5, 150 mM NaCl, 10% glycerol). The purified protein was quantified by SDS-PAGE and Coomassie blue staining using bovine serum albumin (BSA, Thermo Scientific, 23209) as a standard, and stored at −80° C. until use. Human PD-L1 protein with a C-terminal His-tag was purchased from Sino Biological (10084-H08H).

Supported Lipid Bilayer (SLB) Preparation: A glass-bottomed 96-well plate (Cellvis, P96-1.5H-N) was cleaned with 2.5% Hellmanex (Sigma, Z805939) overnight followed by extensive wash with ddH2O. The washed plate was dried with N2 gas, sealed and stored at room temperature until use. Right before use, wells were etched with 6 M NaOH at 50° C. for 1.5 hours and washed with ddH2O and PBS. SUVs (97.9% POPC, 2% DGS-NTA-Ni, and 0.02% PEG5000-PE) were prepared and added to the cleaned wells with 100 μL PBS. The wells were incubated at 50° C. for 2 hours and at room temperature for 30 minutes to form SLBs. The excess SUVs were removed by washing with PBS and the SLBs were functionalized with 3 nM PD-L1-His protein at room temperature for 1 hour. The unbound PD-L1 was removed by washing with PBS and the wells were equilibrated with GUV imaging buffer (100 mM Sodium phosphate, 150 mM NaCl, 1 mM EDTA, 100 mM glucose, pH 7.2).

TIRF Microscopy of GUV-SLB Contact: The JF-PD-1-WALP-CF reconstituted GUVs were mixed with or without 40 μg mL-1 Pembrolizumab and incubated at RT for 10 minutes, and added to the SLB-containing wells with 100 μL GUV imaging buffer. The wells were incubated at room temperature for 10 minutes to let the GUVs settle on the SLB. The fluorescence of GREEN fluorophore and PD-1*JF646 were visualized using Nikon Eclipse Ti TIRF microscope equipped with a 100×Apo TIRF 1.49 NA objective, controlled by the Micro-Manager software. Images were processed using Fiji.9/8/21 5:38:00 PM

Semisynthesis of GFP-WALP and JF-PD-1-WALP in MLVs: Because lipid film hydration may produce multilamellar vesicles at high lipid and peptide concentrations that are necessary for MS and SDS-PAGE analysis, MLVs were used for those experiments. Expressed protein (GFP or PD-1) was diluted in splice buffer to a final concentration of 5 μM and left on ice for 5 minutes. To start the reaction, TM peptide reconstituted into MLVs in splice buffer (40 μL) was added to the expressed protein solution. The reaction was put on a rotator at 37° C. for up to 24 h and samples were sometimes taken from the solution to monitor the reaction progress via SDS-PAGE or LC-ESI-MS. Samples were then visualized by spinning-disk confocal or compound microscopy.

Semisynthesis of GFP-WALP and JF-PD-1-WALP in MLVs: Expressed protein (GFP or PD-1) was added to TM peptide-containing GUVs electroformed in splice buffer to a final concentration of 1 μM. The reaction was put on a rotator at 37° C. for up to 24 h. Samples were then visualized by spinning-disk confocal or compound microscopy.

Microcluster formation of JF-PD-1-WALP reconstituted GUVs.

INFORMAL SEQUENCE LISTING

SEQ ID NO: 1 Cfa^{C}-WALP
VKIISRKSLGTQNVYDIGVGEPHNFLLKNGLVASNCFNGGGWWLALALALALALALAL
ALWWKA
SEQ ID NO: 2 Cfa^{C}-WALP-CF
VKIISRKSLGTQNVYDIGVGEPHNFLLKNGLVASNCFNGGGWWLALALALALALALAL
ALWWKA
SEQ ID NO: 3 GFP-Cfa^{N}-His₆
MKSSRKGEELFTGVVPILVELDGDVNGHKFSVRGEGEGDATNGKLTLKFICTTGKLPVP
WPTLVTTLTYGVQCFARYPDHMKQHDFFKSAMPEGYVQERTISFKDDGTYKTRAEVKF
EGDTLVNRIELKGIDFKEDGNILGHKLEYNFNSHNVYITADKQKNGIKANFKIRHNVEDG
SVQLADHYQQNTPIGDGPVLLPDNHYLSTQSVLSKDPNEKRDHMVLLEFVTAAGITHG
MDELYKGGCLSYDTEILTVEYGFLPIGKIVEERIECTVYTVDKNGFVYTQPIAQWHNRGE
QEVFEYCLEDGSIIRATKDHKFMTTDGQMLPIDEIFERGLDLKQVDGLPHHHHHH
SEQ ID NO: 4 WALP-GFP
MKSSRKGEELFTGVVPILVELDGDVNGHKFSVRGEGEGDATNGKLTLKFICTTGKLPVP
WPTLVTTLTYGVQCFARYPDHMKQHDFFKSAMPEGYVQERTISFKDDGTYKTRAEVKF
EGDTLVNRIELKGIDFKEDGNILGHKLEYNFNSHNVYITADKQKNGIKANFKIRHNVEDG
SVQLADHYQQNTPIGDGPVLLPDNHYLSTQSVLSKDPNEKRDHMVLLEFVTAAGITHG
MDELYKGGCFNGGGWWLALALALALALALALALWWKA
SEQ ID NO: 5 JF-PD-1-Cfa^{N}
GSGSGSFLDSPDRPWNPPTFSPALLVVTEGDNATFTCSFSNTSESFVLNWYRMSPSNQTD
KLAAFPEDRSQPGQDCRFRVTQLPNGRDFHMSVVRARRNDSGTYLCGAISLAPKAQIKE
SLRAELRVTERRAEVPTAHPSPSPRPAGQFQTLVGGCLSYDTEILTVEYGFLPIGKIVEERI
ECTVYTVDKNGFVYTQPIAQWHNRGEQEVFEYCLEDGSIIRATKDHKFMTTDGQMLPID
EIFERGLDLKQVDGLP
SEQ ID NO: 6 JF-PD-1-WALP-CF
GGCFNGGGWWLALALALALALALALALWWKA
SEQ ID NO: 7 Intein Scar Sequence 1
CX
SEQ ID NO: 8 Intein Scar Sequence 2
KKEFECEFL
SEQ ID NO: 9 Intein Scar Sequence 3
ESGSGK
SEQ ID NO: 10 Intein Scar Sequence 4
CFN
SEQ ID NO: 11 Cfa^{N}
CLSYDTEILTVEYGFLPIGKIVEERIECTVYTVDKNGFVYTQPIAQWHNRGEQEVFEYCL
EDGSIIRATKDHKFMTTDGQMLPIDEIFERGLDLKQVDGLPHHHHHH
SEQ ID NO: 12 Cfa^{C}
VKIISRKSLGTQNVYDIGVGEPHNFLLKNGLVASNCFN

### P Embodiments

P Embodiment 1. A first polypeptide comprising a transmembrane domain covalently bound to a C-intein or N-intein.

P Embodiment 2. A vesicle comprising the first polypeptide of P embodiment 1.

P Embodiment 3. A composition comprising the first polypeptide of P embodiment 1 and a second polypeptide covalently bound to a C-intein or N-intein, wherein the if the first polypeptide is bound to a C-intein then the second polypeptides is covalently bound to an N-intein, and wherein if the first polypeptide is bound to a N-intein then the second polypeptides is covalently bound to an C-intein.

P Embodiment 4. A vesicle comprising the composition of P embodiment 3.

P Embodiment 5. A method of synthesis of a transmembrane polypeptide comprising contacting a first polypeptide comprising a transmembrane domain covalently bound to a C-intein with a second polypeptide covalently bound to an N-intein or contacting the first polypeptide covalently bound to a N-intein with the second polypeptide covalently bound to an C-intein.

P Embodiment 6. The method of P embodiment 5, further comprising reconstituting the first polypeptide in a vesicle.

### Embodiments

Embodiment 1. A transmembrane domain covalently bound to a first intein of a split intein pair, wherein said transmembrane domain is embedded within a phospholipid layer.

Embodiment 2. The transmembrane domain of embodiment 1, wherein said phospholipid layer is a lipid vesicle, a nanodisc, a lipid nanoparticle, or a polymersome.

Embodiment 3. The transmembrane domain of embodiments 1 or 2, wherein said first intein is a C-intein.

Embodiment 4. The transmembrane domain of any of embodiments 1 to 3, wherein the split intein is Cfa, PhoRadA, RmaDnaB^{Δ286}, SspDnaB^{Δ275}, SspDnaX, TvoVMA, NpuDnaE, NpuDnaB^{Δ283}, SspGyrB, TerThyX, AceL-TerL, PchPRP8, PfuRIR1-1, Psp-GDBPol-1, PfuRIR1-2, SceVMA^{Δ206}, RmaDnaB^{Δ271}, MtuRecA^{Δ285}, SspDnaB^{Δ274}, gp41-8, SceVMAA²²⁷, IMPDH-1, NrdJ-1, MtuRecA^{Δ297}, gp41-1, AovDnaE, AspDnaE, AvaDnaE, Cra(C5505)DnaE, Csp(CCY0110)DnaE, CwaDnaE, Maer(NIES843)DnaE, Mcht(PCC7420)DnaE, MtuRecA^{Δ300}, NspDnaE, OliDnaE, Sel(PC7942)DnaE, Ssp(PCC7002)DnaE, TerDnaE-3, TelDnaE, TvuDnaE, NeqPol, or TerThyX^{Δ132}.

Embodiment 5. The transmembrane domain of any of embodiments 1 to 4, wherein said transmembrane domain is a PD-1 transmembrane domain, a PD-L1 transmembrane domain, an EGFR transmembrane domain, a proteorhodopsin transmembrane domain, a receptor tyrosine kinase transmembrane domain, a notch receptor transmembrane domain, a hemagglutinin transmembrane domain, a neuraminidase transmembrane domain, an ACE-2 transmembrane domain, a rhomboid protease transmembrane domain, or a WALP peptide.

Embodiment 6. The transmembrane domain of any of embodiments 1 to 5, further comprising a second polypeptide covalently bound to said first intein,

Embodiment 7. The transmembrane domain of embodiment 6, wherein said second polypeptide is covalently bound to a second intein of said split intein pair.

Embodiment 8. The transmembrane domain of embodiment 7, wherein said first intein is a C-intein and said second intein is an N-intein.

Embodiment 9. The transmembrane domain of embodiment 7, wherein said first intein is an N-intein and said second intein is a C-intein.

Embodiment 10. The transmembrane domain of any of embodiments 6 to 9, wherein said second polypeptide an extracellular or intracellular domain of a signaling, receptor, channel, transport, or G-protein coupled receptor (GPCR) membrane protein.

Embodiment 11. A transmembrane domain of any of embodiments 1 to 10, wherein said transmembrane domain is covalently bound to said first intein through a covalent linker.

Embodiment 12. The transmembrane domain of embodiment 11, wherein said linker comprises a peptide linker, wherein said peptide linker is at least 3 amino acids in length.

Embodiment 13. The transmembrane domain of embodiment 12, wherein said peptide linker comprises at least one glycine or one serine residue.

Embodiment 14. A fusion protein comprising a transmembrane domain covalently bound to a biologically active protein domain through a first peptide linker, wherein said transmembrane domain is embedded within a phospholipid layer; and wherein said first peptide linker comprises an intein scar amino acid sequence.

Embodiment 15. The fusion protein of embodiment 14, wherein said intein scar amino acid sequence is the sequence of SEQ ID NO:7, SEQ ID NO:8, SEQ ID ID NO:9, or SEQ ID NO:10.

Embodiment 16. The fusion protein of embodiments 14 or 15, wherein said transmembrane domain covalently is bound to a biologically active protein domain through said first peptide linker and a second linker.

Embodiment 17. The fusion protein of embodiment 16, wherein said second linker is N-terminal to said first peptide linker.

Embodiment 18. The fusion protein of embodiment 16, wherein said second linker is C-terminal to said first peptide linker.

Embodiment 19. The fusion protein of any of embodiments 16 to 18, wherein said second linker comprises a second peptide linker, wherein said second peptide linker is at least 3 amino acids in length.

Embodiment 20. The transmembrane domain of embodiment 19, wherein said second peptide linker comprises at least one glycine or one serine residue.

Embodiment 21. A method of synthesis of a fusion protein, said method comprising: (a) contacting a transmembrane domain with a biologically active protein domain, wherein said transmembrane domain is covalently bound to a first intein of a split intein pair and said transmembrane domain is embedded within a phospholipid layer, wherein said biologically active protein domain is covalently bound to a second intein of said split intein pair, and (b) allowing said first intein to react with said second intein thereby forming said fusion protein.

Embodiment 22. The method of embodiment 21, wherein the reaction of said first and second intein is a transthioesterification reaction.

Embodiment 23. The method of embodiment 21 or 22, wherein said phospholipid layer is a lipid vesicle, a nanodisc, a lipid nanoparticle, or a polymersome.

Embodiment 24. The method of any of embodiments 21 to 23, wherein said first intein is a C-intein or an N-intein.

Embodiment 25. The method of any of embodiments 21 to 24, wherein said second intein is a C-intein or an N-intein.

Embodiment 26. The method of any of embodiments 21 to 25, wherein the split intein is Cfa, PhoRadA, RmaDnaB^{Δ286}, SspDnaB^{Δ275}, SspDnaX, TvoVMA, NpuDnaE, NpuDnaB^{Δ283}, SspGyrB, TerThyX, AceL-TerL, PchPRP8, PfuRIR1-1, Psp-GDBPol-1, PfuRIR1-2, SceVMA^{Δ206}, RmaDnaB^{Δ271}, MtuRecA^{Δ285}, SspDnaB^{Δ274}, gp41-8, SceVMA^{Δ227}, IMPDH-1, NrdJ-1, MtuRecA^{Δ297}, gp41-1, AovDnaE, AspDnaE, AvaDnaE, Cra(C5505)DnaE, Csp(CCY0110)DnaE, CwaDnaE, Maer(NIES843)DnaE, Mcht(PCC7420)DnaE, MtuRecA^{Δ300}, NspDnaE, OliDnaE, Sel(PC7942)DnaE, Ssp(PCC7002)DnaE, TerDnaE-3, TelDnaE, TvuDnaE, NeqPol, or TerThyX^{Δ132}.

Embodiment 27. The method of any of embodiments 21 to 26, wherein said transmembrane domain is covalently bound to said first intein through a first covalent linker.

Embodiment 28. The method of embodiment 27, wherein said first covalent linker comprises a first peptide linker, wherein said first peptide linker is at least 3 amino acids in length.

Embodiment 29. The method of embodiment 28, wherein said first peptide linker comprises at least one glycine or one serine residue.

Embodiment 30. The method of any of embodiments 21 to 29, wherein said biologically active protein domain is covalently bound to said second intein through a second covalent linker.

Embodiment 31. The method of embodiment 30, wherein said second covalent linker comprises a second peptide linker, wherein said second peptide linker is at least 3 amino acids in length.

Embodiment 32. The method of embodiment 31, wherein said second peptide linker comprises at least one glycine or one serine residue.

Embodiment 33. The method of any of embodiments 21 to 32, wherein said transmembrane domain is a PD-1 transmembrane domain, a PD-L1 transmembrane domain, an EGFR transmembrane domain, a proteorhodopsin transmembrane domain, a receptor tyrosine kinase transmembrane domain, a notch receptor transmembrane domain, a hemagglutinin transmembrane domain, a neuraminidase transmembrane domain, an ACE-2 transmembrane domain, a rhomboid protease transmembrane domain, or a WALP peptide.

Embodiment 34. The method of any of embodiments 21 to 33, wherein said biologically active protein domain is an extracellular or intracellular domain of a signaling, receptor, channel, transport, or G-protein coupled receptor (GPCR) membrane protein.

Embodiment 35. A kit composition comprising a transmembrane domain covalently bound to a first intein of a split intein pair, wherein said transmembrane domain is embedded within a phospholipid layer.

What is claimed is:

1. A transmembrane domain covalently bound to a first intein of a split intein pair, wherein said transmembrane domain is embedded within a phospholipid layer.

2. The transmembrane domain of claim 1, wherein said phospholipid layer is a lipid vesicle, a nanodisc, a lipid nanoparticle, or a polymersome.

3. The transmembrane domain of claim 1, wherein said first intein is a C-intein.

4. The transmembrane domain of claim 1, wherein the split intein is a C-intein or an N-intein from one of the following inteins: Cfa, PhoRadA, RmaDnaB^{Δ286}, SspDnaB^{Δ275}, SspDnaX, TvoVMA, NpuDnaE, NpuDnaB^{Δ283}, SspGyrB, TerThyX, AceL-TerL, PchPRP8, PfuRIR1-1, Psp-GDBPol-1, PfuRIR1-2, SceVMA^{Δ206}, RmaDnaB^{Δ27}1, MtuRecA^{Δ285}, SspDnaB^{Δ274}, gp41-8, SceVMAA²²⁷, IMPDH-1, NrdJ-1, MtuRec^{Δ297}, gp41-1, AovDnaE, AspDnaE, AvaDnaE, Cra(C5505)DnaE, Csp(CCY0110)DnaE, CwaDnaE, Maer(NIES843)DnaE, Mcht(PCC7420)DnaE, MtuRecA^{Δ300}, NspDnaE, OliDnaE, Sel(PC7942)DnaE, Ssp(PCC7002)DnaE, TerDnaE-3, TelDnaE, TvuDnaE, NeqPol, or TerThyX^{Δ132}.

5. The transmembrane domain of claim 1, wherein said transmembrane domain is a PD-1 transmembrane domain, a PD-L1 transmembrane domain, an EGFR transmembrane domain, a proteorhodopsin transmembrane domain, a receptor tyrosine kinase transmembrane domain, a notch receptor transmembrane domain, a hemagglutinin transmembrane domain, a neuraminidase transmembrane domain, an ACE-2 transmembrane domain, a rhomboid protease transmembrane domain, or a WALP peptide.

6. The transmembrane domain of claim 1, further comprising a second polypeptide covalently bound to said first intein.

7. The transmembrane domain of claim 6, wherein said second polypeptide is covalently bound to a second intein of said split intein pair.

8. The transmembrane domain of claim 7, wherein said first intein is a C-intein and said second intein is an N-intein.

9. The transmembrane domain of claim 7, wherein said first intein is an N-intein and said second intein is a C-intein.

10. The transmembrane domain of claim 6, wherein said second polypeptide is an extracellular or intracellular domain of a signaling, receptor, channel, transport, or G-protein coupled receptor (GPCR) membrane protein.

11. A transmembrane domain of claim 1, wherein said transmembrane domain is covalently bound to said first intein through a covalent linker.

12. The transmembrane domain of claim 11, wherein said linker comprises a peptide linker, wherein said peptide linker is at least 3 amino acids in length.

13. The transmembrane domain of claim 12, wherein said peptide linker comprises at least one glycine or one serine residue.

14. A fusion protein comprising a transmembrane domain covalently bound to a biologically active protein domain through a first peptide linker, wherein said transmembrane domain is embedded within a phospholipid layer; and wherein said first peptide linker comprises an intein scar amino acid sequence.

15. The fusion protein of claim 14, wherein said intein scar amino acid sequence is the sequence of SEQ ID NO:7, SEQ ID NO:8, SEQ ID NO:9, or SEQ ID NO:10.

16. The fusion protein of claim 14, wherein said transmembrane domain covalently is bound to a biologically active protein domain through said first peptide linker and a second linker.

17. The fusion protein of claim 16, wherein said second linker is N-terminal to said first peptide linker.

18. The fusion protein of claim 16, wherein said second linker is C-terminal to said first peptide linker.

19. The fusion protein of claim 16, wherein said second linker comprises a second peptide linker, wherein said second peptide linker is at least 3 amino acids in length.

20. The transmembrane domain of claim 19, wherein said second peptide linker comprises at least one glycine or one serine residue.

21. A method of synthesis of a fusion protein, said method comprising:
(a) contacting a transmembrane domain with a biologically active protein domain,
wherein said transmembrane domain is covalently bound to a first intein of a split intein pair and said transmembrane domain is embedded within a phospholipid layer,
wherein said biologically active protein domain is covalently bound to a second intein of said split intein pair; and
(b) allowing said first intein to react with said second intein thereby forming said fusion protein.

22. The method of claim 21, wherein the reaction of said first and second intein is a transthioesterification reaction.

23. The method of claim 21, wherein said phospholipid layer is a lipid vesicle, a nanodisc, a lipid nanoparticle, or a polymersome.

24. The method of claim 21, wherein said first intein is a C-intein or an N-intein.

25. The method of claim 21, wherein said second intein is a C-intein or an N-intein.

26. The method of claim 21, wherein the split intein is a C-intein or N-intein from Cfa, PhoRadA, RmaDnaB^{Δ286}, SspDnaB^{Δ275}, SspDnaX, TvoVMA, NpuDnaE, NpuDnaB^{Δ283}, SspGyrB, TerThyX, AceL-TerL, PchPRP8, PfuRIR1-1, Psp-GDBPol-1, PfuRIR1-2, SceVMA^{Δ206}, RmaDnaB^{Δ27}1, MtuRecA^{Δ285}, SspDnaB^{Δ274}, gp41-8, SceVMAA²²⁷, IMPDH-1, NrdJ-1, MtuRecA^{Δ297}, gp41-1, AovDnaE, AspDnaE, AvaDnaE, Cra(C5505)DnaE, Csp(CCY0110)DnaE, CwaDnaE, Maer(NIES843)DnaE, Mcht(PCC7420)DnaE, MtuRecA^{Δ300}, NspDnaE, OliDnaE, Sel(PC7942)DnaE, Ssp(PCC7002)DnaE, TerDnaE-3, TelDnaE, TvuDnaE, NeqPol, or TerThyX^{Δ132}.

27. The method of claim 21, wherein said transmembrane domain is covalently bound to said first intein through a first covalent linker.

28. The method of claim 27, wherein said first covalent linker comprises a first peptide linker, wherein said first peptide linker is at least 3 amino acids in length.

29. The method of claim 28, wherein said first peptide linker comprises at least one glycine or one serine residue.

30. The method of claim 21, wherein said biologically active protein domain is covalently bound to said second intein through a second covalent linker.

31. The method of claim 30, wherein said second covalent linker comprises a second peptide linker, wherein said second peptide linker is at least 3 amino acids in length.

32. The method of claim 31, wherein said second peptide linker comprises at least one glycine or one serine residue.

33. The method of claim 21, wherein said transmembrane domain is a PD-1 transmembrane domain, a PD-L1 transmembrane domain, an EGFR transmembrane domain, a proteorhodopsin transmembrane domain, a receptor tyrosine kinase transmembrane domain, a notch receptor transmembrane domain, a hemagglutinin transmembrane domain, a neuraminidase transmembrane domain, an ACE-2 transmembrane domain, a rhomboid protease transmembrane domain, or a WALP peptide.

34. The method of claim 21, wherein said biologically active protein domain is an extracellular or intracellular domain of a signaling, receptor, channel, transport, or G-protein coupled receptor (GPCR) membrane protein.

35. A kit composition comprising a transmembrane domain covalently bound to a first intein of a split intein pair, wherein said transmembrane domain is embedded within a phospholipid layer.


SEQUENCE LISTING (12 sequences)

SEQ ID NO: 1 - protein, 64 aa, synthetic construct
VKIISRKSLGTQNVYDIGVGEPHNFLLKNGLVASNCFNGGGWWLALALALALALALALALWWKA

SEQ ID NO: 2 - protein, 64 aa, synthetic construct; identical to SEQ ID NO: 1

SEQ ID NO: 3 - protein, 350 aa, synthetic construct
MKSSRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPHHHHHH

SEQ ID NO: 4 - protein, 272 aa, synthetic construct
MKSSRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGCFNGGGWWLALALALALALALALALWWKA

SEQ ID NO: 5 - protein, 256 aa, synthetic construct
GSGSGSFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 6 - protein, 31 aa, synthetic construct
GGCFNGGGWWLALALALALALALALALWWKA

SEQ ID NO: 7 - skipped

SEQ ID NO: 8 - protein, 9 aa, synthetic construct
KKEFECEFL

SEQ ID NO: 9 - protein, 6 aa, synthetic construct
ESGSGK

SEQ ID NO: 10 - skipped

SEQ ID NO: 11 - protein, 107 aa, synthetic construct
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPHHHHHH

SEQ ID NO: 12 - protein, 38 aa, synthetic construct
VKIISRKSLGTQNVYDIGVGEPHNFLLKNGLVASNCFN